(19) United States
(12) Patent Application Publication
(10) Pub. No.: US 2025/0129139 A1
(43) Pub. Date: Apr. 24, 2025

(54) CHIMERIC ANTIGEN RECEPTOR T CELLS TARGETING gD AND ONCOLYTIC VIRUSES FOR CANCER THERAPY AND TREATMENT OF HSV

(71) Applicant: City of Hope, Duarte, CA (US)

(72) Inventors: Saul J. Priceman, Valley Village, CA (US); Yuman Fong, Duarte, CA (US); Anthony K. Park, Duarte, CA (US)

(21) Appl. No.: 18/682,713

(22) PCT Filed: Aug. 9, 2022
(86) PCT No.: PCT/US2022/074716

§ 371 Date: Feb. 9, 2024

Related U.S. Application Data
(60) Provisional application No. 63/231,203, filed on Aug. 9, 2021.

(51) Int. Cl. C07K 16/08; A61K 35/763; A61K 39/395; A61K 40/11; A61K 40/31; A61K 40/46; A61P 35/00; C12N 5/0783

(52) U.S. Cl. CPC C07K 16/087; A61K 35/763; A61K 39/39558; A61K 40/11; A61K 40/31; A61K 40/46; A61P 35/00; C12N 5/0636; C07K 2317/622; C07K 2317/73; C07K 2319/02; C07K 2319/03; C12N 2510/00

(57) ABSTRACT

Chimeric antigen receptors (CAR) targeted to glycoprotein D (“gD”) and immune cells (e.g., T cells and NK cells) expressing such CAR are described. Nucleic acids encoding a gD-CAR and immune cells (e.g., T cells and NK cells) comprising such nucleic acids are also described. Methods of making and using (e.g., treating a cancer and treating a Herpes Simplex Virus infection) such immune cells with or without combining treatment with an oncolytic Herpes Simplex Virus are also disclosed.

### CLAIM OF PRIORITY

This application claims the benefit of U.S. Provisional Application Ser. No. 63/231,203, filed on Aug. 9, 2021. The entire contents of the foregoing are incorporated herein by reference.

### TECHNICAL FIELD

This disclosure relates to treating cancer using anti-glycoprotein D CAR immune cell therapy with or without an oncolytic Herpes Simplex Virus as well as methods for treating infection by Herpes Simplex Virus with an anti-glycoprotein D CAR immune cell therapy.

### BACKGROUND

Chimeric antigen receptor (CAR) engineered T cells have energized the field of cancer immunotherapy with their proven ability to treat hematological malignancies, yet the success of CAR T cells against solid tumors has been limited. The relative lack of success of CAR T cell therapy against solid tumors is likely due to a variety of factors, including: the antigen heterogeneity of solid tumors, the difficulty trafficking CAR T cells to solid tumors, and paucity of tumor selective targets. Thus, there is a need for CAR T cell therapies that are effective against solid tumors.

### SUMMARY

The present disclosure is based, at least in part, on the discovery that treatment with cells expressing a chimeric antigen receptors (“CAR”) targeted to glycoprotein D (“gD”) can eliminate cells expressing glycoprotein D. The gD CAR, expressed, for example, by a T cell, can be administrated in combination with an oncolytic herpes simplex virus (“oHSV”) to kill solid tumor cells. The oHSV can infect solid tumor cells and sufficiently direct gD expression by an infected cells to permit killing by T cells or other immune cells expressing a gD CAR.

Accordingly, aspects of the present disclosure provide nucleic acid molecules encoding a chimeric antigen receptors. A useful nucleic acid molecule encodes a chimeric antigen receptor, wherein the chimeric antigen receptor comprises: (i) an scFv that binds HSV envelope glycoprotein D; (ii) a spacer domain; (iii) a transmembrane domain; (iv) a costimulatory domain; and (v) a CD35ζ signaling domain. In various embodiments: the spacer region comprises 5-300 amino acids; the spacer comprises an IgG hinge region; the scFv comprises: a light chain CDR1 comprising RASQSVTSSQLA, a light chain CDR2 comprising GASNRAT, a light chain CDR3 comprising QQYGSSPT, a heavy chain CDR1 comprising TYGVS, a heavy chain CDR2 comprising RTIPLFGKTDYAQKFQG, and a heavy chain CDR3 comprising DLTTLTSYNWWDL; the scFV comprises: (a) a light chain variable domain that is at least 90%, 95% or 98% identical to: EIVLTQSPGTLSLSPGERATLSCRASQSVTSSQLAWYQQKPGQAPRLLISGASNRATGI PDRFSGSGSGTDFTLTISRLEPEDFAVYYCQQYGSSPTFGGGTKVEIKR; (b) a heavy chain variable domain that is at least 90%, 95% or 98% identical to: QVTLKQSGAEVKKPGSSVKVSCTASGGTLRTYGVSWVRQAPGQGLEWLGRTIPLFG KTDYAQKFQGRVTITADKSMDTSFMELTSLTSEDTAVYYCARDLTTLTSYNWWDL WGQGTLVTVSS; or (c) a light chain variable domain that is at least 90%, 95% or 98% identical to:
EIVLTQSPGTLSLSPGERATLSCRASQSVTSSQLAWYQQKPGQAPRLLISGASNRATGI PDRFSGSGSGTDFTLTISRLEPEDFAVYYCQQYGSSPTFGGGTKVEIKR; and a heavy chain variable domain that is at least 90%, 95% or 98% identical to:
QVTLKQSGAEVKKPGSSVKVSCTASGGTLRTYGVSWVRQAPGQGLEWLGRTIPLFG KTDYAQKFQGRVTITADKSMDTSFMELTSLTSEDTAVYYCARDLTTLTSYNWWDL WGQGTLVTVSS; the scFV comprises: a light chain variable domain comprising EIVLTQSPGTLSLSPGERATLSCRASQSVTSSQLAWYQQKPGQAPRLLISGASNRATGI PDRFSGSGSGTDFTLTISRLEPEDFAVYYCQQYGSSPTFGGGTKVEIKR; and a heavy chain variable domain comprising
QVTLKQSGAEVKKPGSSVKVSCTASGGTLRTYGVSWVRQAPGQGLEWLGRTIPLFG KTDYAQKFQGRVTITADKSMDTSFMELTSLTSEDTAVYYCARDLTTLTSYNWWDL WGQGTLVTVSS; and the spacer region comprises an amino acid sequence selected from the group consisting of SEQ ID NOs: 24-34; the transmembrane domain selected from the group consisting of: a CD4 transmembrane domain, a CD8 transmembrane domain, a CD28 transmembrane domain, and a CD32 transmembrane domain; the costimulatory domain selected from the group consisting of: a CD28 costimulatory domain, a 41-BB costimulatory domain, an OX40 costimulatory domain, and a 2B4 costimulatory domain; the chimeric antigen receptor can comprises or consist of the amino acid sequence of any one of SEQ ID NO: 48-49 or 57-126; and the chimeric antigen receptor can comprises or consist of the amino acid sequence of any one of SEQ ID NO: 48-49 or 57-126 with 1, 2, 3, 4 or 5 single amino acid substitutions or deletions.

A number of useful gD-targeting sequences could be used in the methods and compositions disclosed herein, including any scFv, V_{H} and V_{L} domains, and CDR sequences disclosed in the following non-limiting list:
E317 (Lee et al. 2013 Structural basis for the antibody neutralization of herpes simplex virus. Acta Cystallography Section D Biological Crystallography; 69:1935-1945; U.S. Pat. No. 8,252,906);
M27f (Du R et al. 2017 Antiviral Res147: 131-141;
III-114-4, III-174-1, III-255-2, and II-529-3 (Fuller et al. 1987 Proc Natl Acad Sci USA 84:5454-5458);
LP14 (Millipore-Sigma Catalog No. MABF1975);
2C10 (Antibodies-Online; Catalog No. ABIN265572);
NB100-63170 (Novus Biologicals);
SKM/56 (HSV-1; ImmuQuest Ltd; Catalog No. IQ413);
DCABY-3980 (Creative DiagnosticsDMABT-Z60828); and
Para et al. 1985 J. Virology 55:483-488.

Also disclosed are nucleic acid molecules encoding a chimeric antigen receptor, wherein the chimeric antigen receptor comprises: a scFv comprising SEQ ID NO: 2; a spacer comprising a sequence selected from the group consisting of: SEQ ID NOs: 24-34; a transmembrane domain comprising a sequence selected from the group consisting of SEQ ID NOs: 15-23; a costimulatory domain comprising a sequence selected from the group consisting of SEQ ID NOs: 36-40, and a CD35 signaling domain comprising SEQ ID NO: 35.

Also disclosed are immune cells harboring any nucleic acid molecule described herein.

Also disclosed are methods of treating a patient infected with HSV, the method comprising administering a therapeutically effective amount of immune cells described herein expressing a gD CAR.

Also described are methods of treating cancer, comprising administering an oncolytic HSV (oHSV) and a therapeutically effective amount of immune cells described herein expressing a gD CAR.

In various embodiments the oHSV: lacks a functional ICP34.5 encoding gene, lacks a functional ICP47 encoding gene and comprises a gene encoding human GM-CSF; the oHSV is talimogene laherparepvec; the oHSV is selected from the group consisting of: HF-10 (Takara Bio, Inc.; lacks UL43, UL49.5, UL55, UL56, and LAT), HSV-1716 (Virttu Biologics; lacks ICP34.5), G207 (Medigene; lacks ICP34.5 and ICP6 (substituted with LacZ), M032 (Acttis, Inc), and G47Δ (Daiichi Sankyo Company; lacks ICP34.5, ICP6 and ICP47).

In various embodiments the method for treating cancer also comprises administering an effective amount of an anti-PD-1 antibody (e.g., nivolumab, lambrolizumab, CT-011 or AMP-224) or anti-CTLA-4 antibody (e.g., ipilimumab).

### I. Anti-gD CAR

A chimeric antigen receptor (CAR) refers to an artificial immune cell receptor that is engineered to recognize and bind to a surface antigen. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR T cells the ability to recognize an antigen independent of antigen processing, thereby bypassing a major mechanism of tumor escape. A CAR can also be expressed by other immune effector cells, including but not limited to natural killer CAR (“NK CAR”) and directed NK cell killing to cells expressing the target of the CAR.

There are various generations of CARs, each of which contains different components. First generation CARs join an antibody-derived scFv to the CD35 intracellular signaling domain of the T cell receptor through a spacer region (also called a hinge domain) and a transmembrane domain. Second generation CARs incorporate an additional co-stimulatory domain (e.g., CD28, 4-BB, or ICOS) to supply a co-stimulatory signal. Third generation CARs contain two co-stimulatory domains (e.g., a combination of CD27, CD28, 4-1BB, ICOS, or OX40) fused with the TCR CD35 chain. Any generation of CAR is within the scope of the present disclosure.

The gD CAR described herein are fusion proteins comprising an extracellular domain that recognizes herpes simplex virus (“HSV”) gD (e.g., a single chain fragment (scFv) of an antibody or other antibody fragment), a spacer, a transmembrane domain, at least one co-stimulatory domain and an intracellular domain comprising a signaling domain of the T cell receptor (TCR) complex (e.g., CD35ζ). A CAR is often fused to a signal peptide at the N-terminus for surface expression.

Provided herein are HSV glycoprotein D (gD) targeted CARs (also called “gD CAR”). The gD CAR can comprise an anti-gD scFv specific for gD.

HSV gD (human herpes simplex virus 1; GenBank YP_009137141) has the sequence:

(SEQ ID NO: 1)

| | |
|---|---|
| 1 | mggaaarlga vilfvvivgl hgvrgkyalv daslkmadpn |
| | rfrgkdlpvl dqltdppgvr |
| 61 | rvyhiqaglp dpfqppslpi tvyyavlera crsvllnaps |
| | eapqivrgas edvrkqpynl |
| 121 | tiawfrmggn caipitvmey tecsynkslg acpirtqprw |
| | nyydsfsavs ednlgflmha |
| 181 | pafetagtyl rlvkindwte itqfilehra kgsckyalpl |
| | rippsaclsp qayqqgvtvd |
| 241 | sigmlprfip enqrtvavys lkiagwhgpk apytstllpp |
| | elsetpnatq pelapedped |
| 301 | salledpvgt vapqippnwh ipsiqdaatp yhppatpnnm |
| | gliagavggs llaalvicgi |
| 361 | vywmrrhtqk apkrirlphi reddqpsshq plfy |

### (a) Antigen Binding Extracellular Domain

The antigen binding extracellular domain is the region of a CAR polypeptide that is exposed to the extracellular fluid when the CAR is expressed on the cell surface. The antigen binding extracellular domain is specific to a target antigen of interest such as a tumor antigen (e.g., gD). In some examples, the antigen binding domain comprises a scFv, which includes an antibody heavy chain variable region (V_{H}) and an antibody light chain variable region (V_{L}). The scFV fragment retains the antigen binding specificity of the parent antibody, from which the scFv fragment is derived. The V_{H} and V_{L} domains can be in either orientation (i.e., V_{H}-V_{L} Or V_{L}-V_{H}). In some examples, the V_{H} and V_{L} are linked via a peptide linker, which can include hydrophilic residues with stretches of glycine and serine for flexibility as well as stretches of glutamate and lysine for improved solubility. In some embodiments, the scFv can comprise humanized V_{H} and/or V_{L} domains. In some examples, a signal peptide can be located at the N-terminus to facilitate cell surface expression.

In some cases it is desirable for the scFv targeted to human gD to bind to HSV1 gD and HSV2 gD.

In some embodiments, the scFv targeted to gD
comprises the amino acid sequence:
(SEQ ID NO: 2)
QVTLKQSGAEVKKPGSSVKVSCTASGGTLRTYGVSWVRQAPGQGLEWLGR
TIPLFGKTDYAQKFQGRVTITADKSMDTSFMELTSLTSEDTAVYYCARDL
TTLTSYNWWDLWGQGTLVTVSSGGGGSGGGGSGGGGSEIVLTQSPGTLSL
SPGERATLSCRASQSVTSSQLAWYQQKPGQAPRLLISGASNRATGIPDRF
SGSGSGTDFTLTISRLEPEDFAVYYCQQYGSSPTFGGGTKVEIKR with
up to 5 or up to 10 single amino acid
substitutions.
The signal sequence is:
(SEQ ID NO: 3)
MLLLVTSLLLCELPHPAFLLIP
The heavy chain variable domain (VH) comprises the
sequence:
(SEQ ID NO: 8)
QVTLKQSGAEVKKPGSSVKVSCTASGGTLRTYGVSWVRQAPGQGLEWLGR
TIPLFGKTDYAQKFQGRVTITADKSMDTSFMELTSLTSEDTAVYYCARDL
TTLTSYNWWDLWGQGTLVTV
The light chain variable domain (VL) comprises the
sequence:
(SEQ ID NO: 4)
EIVLTQSPGTLSLSPGERATLSCRASQSVTSSQLAWYQQKPGQAPRLLIS
GASNRATGIPDRFSGSGSGTDFTLTISRLEPEDFAVYYCQQYGSSPTFGG
GTKVEIKR

The V_{H} can precede the V_{L} and a linker comprising the sequence SSGGGGSGGGGSGGGGS (SEQ ID NO:12) can be located between the V_{H} domain and the V_{L} domain. The V_{L} can precede the V_{H} and a linker comprising the sequence SSGGGGSGGGGSGGGGS (SEQ ID NO:12) can be located between the V_{L} domain and the V_{H} domain.

The scFv can include a HC CDR1 comprising the amino acid sequence TYGVS (SEQ ID NO: 9) or GGTLRTYGVS (SEQ ID NO:41); a HC CDR2 comprising the amino acid sequence: RTIPLFGKTDYAQKFQG (SEQ ID NO: 10); a HC CDR3 comprising the amino acid sequence: DLTTLTSYNWWDL (SEQ ID NO: 11); a LC CDR1 comprising the amino acid sequence RASQSVTSSQLA (SEQ ID NO: 5); a LC CDR2 comprising the amino acid sequence: GASNRAT (SEQ ID NO: 6); and a LC CDR3 comprising the amino acid sequence: QQYGSSPT (SEQ ID NO: 7).

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In certain embodiments, the gD scFv comprises a light chain variable region that is at least 95% identical to or includes up to 5 single amino acid substitutions compared to: SEQ ID NO: 4. In certain embodiments, the gD scFv comprises a light chain variable region that comprises a CDR1 comprising: SEQ ID NO: 5, a CDR2 comprising SEQ ID NO: 6; and a CDR3 comprising SEQ ID NO: 7 and is overall at least 95, 96, 97, 98, or 99% identical to SEQ ID NO: 4.

In certain embodiments, the gD scFv comprises a heavy chain variable region that is at least 95% identical to or includes up to 5 single amino acid substitutions compared to: SEQ ID NO: 8. In certain embodiments, the gD scFv comprises a heavy chain variable region that comprises a CDR1 comprising: SEQ ID NO: 9, a CDR2 comprising SEQ ID NO: 10; and a CDR3 comprising SEQ ID NO: 11 and is overall at least 95, 96, 97, 98 or 99% identical to SEQ ID NO: 8.

In certain embodiments, the gD scFv comprises a light chain variable region comprising SEQ ID NO: 4 and a heavy chain variable region comprising SEQ ID NO: 8 joined by a linker of 5-20 amino acids. In some embodiments, a useful flexible linker is 1, 2, 3, 4, 5, 6, 7, 8, 9, or 10 repeats of the sequence GGGS (SEQ ID NO: 13). In some embodiments, a useful flexible linker is 1, 2, 3, 4, 5, 6, 7, 8, 9, or 10 repeats of the sequence GGGGS (SEQ ID NO: 14). In some embodiments, the light chain variable domain is amino terminal to the heavy chain variable domain in other cases it is carboxy terminal to the heavy chain variable domain. In some cases the linker comprises the sequence SSGGGGSGGGGSGGGGS (SEQ ID NO:12).

In certain embodiments, the gD scFv comprises an amino acid sequence that is 90, 95, 96, 97, 98, 99, or 100% identical to SEQ ID NO:2. In certain embodiments, the gD scFv comprises SEQ ID NO:2 with up to 1, 2, 3, 4, or 5 amino acid substitutions, wherein the substitutions are not in the CDR region and/or the substitutions are conservative.

In certain embodiments, the scFv includes a HC CDR1 comprising the amino acid sequence TYGVS (SEQ ID NO: 9) or GGTLRTYGVS (SEQ ID NO:41); a HC CDR2 comprising the amino acid sequence: RTIPLFGKTDYAQKFQG (SEQ ID NO: 10); a HC CDR3 comprising the amino acid sequence: DLTTLTSYNWWDL (SEQ ID NO: 11); a LC CDR1 comprising the amino acid sequence RASQSVTSSQLA (SEQ ID NO: 5); a LC CDR2 comprising the amino acid sequence: GASNRAT (SEQ ID NO: 6); and a LC CDR3 comprising the amino acid sequence: QQYGSSPT (SEQ ID NO: 7) and is overall 90, 95, 96, 97, 98, 99, or 100% identical to SEQ ID NO:2.

In certain embodiments, the gD scFv comprises SEQ ID NO:2 with up to 1, 2, 3, 4, or 5 amino acid substitutions and includes a HC CDR1 comprising the amino acid sequence TYGVS (SEQ ID NO: 9) or GGTLRTYGVS (SEQ ID NO:41); a HC CDR2 comprising the amino acid sequence: RTIPLFGKTDYAQKFQG (SEQ ID NO: 10); a HC CDR3 comprising the amino acid sequence: DLTTLTSYNWWDL (SEQ ID NO: 11); a LC CDR1 comprising the amino acid sequence RASQSVTSSQLA (SEQ ID NO: 5); a LC CDR2 comprising the amino acid sequence: GASNRAT (SEQ ID NO: 6); and a LC CDR3 comprising the amino acid sequence: QQYGSSPT (SEQ ID NO: 7)

### (b) Transmembrane Domain

Any CAR and polypeptides disclosed herein can contain a transmembrane domain, which can be a hydrophobic alpha helix that spans the membrane. As used herein, a transmembrane domain refers to any protein structure that is thermodynamically stable in a cell membrane, preferably a eukaryotic cell membrane.

The transmembrane domain of a CAR as provided herein can be a CD28 transmembrane domain having the sequence: FWVLVVVGGVLACYSLLVTVAFIIFWV (SEQ ID NO: 16). Other transmembrane domains can be used including those shown below in Table 1.

TABLE 1
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 15) |
| CD28 | NM_006139 | 27 aa | FWVLVVVGGVLACYSLLVTVAFI |
| | | | IFWV (SEQ ID NO: 16) |
| CD28 | NM_006139 | 28 aa | MFWVLVVVGGVLACYSLLVTVAF |
| (M) | | | IIFWV (SEQ ID NO: 17) |
| CD4 | M35160 | 22 aa | MALIVLGGVAGLLLFIGLGIFF |
| | | | (SEQ ID NO: 18) |
| CD8tm | NM_001768 | 21 aa | IYIWAPLAGTCGVLLLSLVIT |
| | | | (SEQ ID NO: 19) |
| CD8tm2 | NM_001768 | 23 aa | IYIWAPLAGTCGVLLLSLVITLY |
| | | | (SEQ ID NO: 20) |
| CD8tm3 | NM_001768 | 24 aa | IYIWAPLAGTCGVLLLSLVITLYC |
| | | | (SEQ ID NO: 21) |
| 4-1BB | NM_001561 | 27 aa | IISFFLALTSTALLFLLFF |
| | | | LTLRFSVV (SEQ ID NO: 22) |
| NKG2D | NM_007360 | 21 aa | PFFFCCFIAVAMGIRFIIMVA |
| | | | (SEQ ID NO: 23) |

### (c) Spacer Region

Any CAR or polypeptide described herein can include a spacer region located between the gD targeting domain (i.e., a gD targeted scFv or variant thereof) and the transmembrane domain. Without being bound by theory, the spacer region can function to provide flexibility to the CAR, or domains thereof, or to prevent steric hindrance of the CAR, or domains thereof. A variety of different spacers can be used. Some of them include at least portion of a human Fc region, for example a hinge portion of a human Fc region or a CH3 domain, or variants thereof. Table 2 below provides various spacers that can be used in the CARs or polypeptides described herein.

TABLE 2
Examples of Spacers

| Name | Length | Sequence |
|---|---|---|
| a3 | 3 aa | AAA |
| linker | 10 aa | GGGSSGGGSG (SEQ ID NO: 24) |
| IgG4 hinge (S→P) | 12 aa | ESKYGPPCPPCP (SEQ ID NO: 25) |
| (S228P) | | |
| IgG4 hinge | 12 aa | ESKYGPPCPSCP (SEQ ID NO: 26) |
| IgG4 hinge (S228P) + | 22 aa | ESKYGPPCPPCPGGGSSGGGSG (SEQ ID NO: |
| linker | | 27) |
| CD28 hinge | 39 aa | IEVMYPPPYLDNEKSNGTIIHVKGKHLCPSPLF |
| | | PGPSKP (SEQ ID NO: 28) |
| CD8 hinge-48 aa | 48 aa | AKPTTTPAPRPPTPAPTIASQPLSLRPEACRPAA |
| | | GGAVHTRGLDFACD (SEQ ID NO: 29) |
| CD8 hinge-45 aa | 45 aa | TTTPAPRPPTPAPTIASQPLSLRPEACRPAAGGA |
| | | VHTRGLDFACD (SEQ ID NO: 30) |
| IgG4(HL-CH3) | 129 aa | ESKYGPPCPPCPGGGSSGGGSGGQPREPQVYT |
| Also called IgG4(HL- | | LPPSQEEMTKNQVSLTCLVKGFYPSDIAVEWE |
| ΔCH2) | | SNGQPENNYKTTPPVLDSDGSFFLYSRLTVDK |
| (includes S228P in | | SRWQEGNVFSCSVMHEALHNHYTQKSLSLSL |
| hinge) | | GK (SEQ ID NO: 31) |
| IgG4(L235E, N297Q) | 229 aa | ESKYGPPCPSCPAPEFEGGPSVFLFPPKPKDTL |
| | | MISRTPEVTCVVVDVSQEDPEVQFNWYVDGV |
| | | EVHNAKTKPREEQFQSTYRVVSVLTVLHQDW |
| | | LNGKEYKCKVSNKGLPSSIEKTISKAKGQPREP |
| | | QVYTLPPSQEEMTKNQVSLTCLVKGFYPSDIA |
| | | VEWESNGQPENNYKTTPPVLDSDGSFFLYSRL |
| | | TVDKSRWQEGNVFSCSVMHEALHNHYTQKSL |
| | | SLSLGK (SEQ ID NO: 32) |
| IgG4(S228P, | 229 aa | ESKYGPPCPPCPAPEFEGGPSVFLFPPKPKDTL |
| L235E, N297Q) | | MISRTPEVTCVVVDVSQEDPEVQFNWYVDGV |
| | | EVHNAKTKPREEQFQSTYRVVSVLTVLHQDW |
| | | LNGKEYKCKVSNKGLPSSIEKTISKAKGQPREP |
| | | QVYTLPPSQEEMTKNQVSLTCLVKGFYPSDIA |
| | | VEWESNGQPENNYKTTPPVLDSDGSFFLYSRL |
| | | TVDKSRWQEGNVFSCSVMHEALHNHYTQKSL |
| | | SLSLGK (SEQ ID NO: 33) |
| IgG4(CH3) | 107 aa | GQPREPQVYTLPPSQEEMTKNQVSLTCLVKGF |
| Also called | | YPSDIAVEWESNGQPENNYKTTPPVLDSDGSF |
| IgG4(ΔCH2) | | FLYSRLTVDKSRWQEGNVFSCSVMHEALHNH |
| | | YTQKSLSLSLGK (SEQ ID NO: 34) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO: 26) or ESKYGPPCPPCP (SEQ ID NO: 25). The hinge/linger region can also comprise the sequence ESKYGPPCPPCP (SEQ ID NO: 25) followed by the linker sequence GGGSSGGGSG (SEQ ID NO: 24) followed by IgG4 CH3 sequence GQPREPQVYTLPPSQEEMTKNQVSLTCLVKGFYPSDIA VEWESNGQPENNYKTTPPV LDSDGSFFLYSRLTVDKSRWQEGNVFSCSVMHEALHNHYTQKSLSLSLGK (SEQ ID NO: 34). Thus, the entire linker/spacer region can comprise the sequence: ESKYGPPCPPCPGGGSSGGGSGGQPREPQVYTLPPSQEEMTKNQVSLTCLVKGFYPS DIAVEWESNGQPENNYKTTPPVLDSDGSFFLYSRLTVDKSRWQEGNVFSCSVMHEA LHNHYTQKSLSLSLGK (SEQ ID NO: 31). In some cases, the spacer has 1, 2, 3, 4, or 5 single amino acid changes (e.g., conservative changes) compared to SEQ ID NO: 31. In some cases, the IgG4 Fc hinge/linker region that is mutated at two positions (L235E; N297Q) in a manner that reduces binding by Fc receptors (FcRs).

### (d) Intracellular Signaling Domains

Any of the CAR constructs described herein contain one or more intracellular signaling domains (e.g., CD3 ζ, and optionally one or more co-stimulatory domains), which are the functional end of the receptor. Following antigen recognition, receptors cluster and a signal is transmitted to the cell.

CD32ζ is the cytoplasmic signaling domain of the T cell receptor complex. CD32ζ contains three immunoreceptor tyrosine-based activation motifs (ITAMs), which transmit an activation signal to the T cell after the T cell is engaged with a cognate antigen. In some cases, CD3ζ provides a primary T cell activation signal but not a fully competent activation signal, which requires a co-stimulatory signal.

Accordingly, in some examples, the CAR polypeptides disclosed herein may further comprise one or more co-stimulatory signaling domains in addition to CD3 ζ. For example, the co-stimulatory domain CD28 and/or 4-1BB can be used to transmit a proliferative/survival signal together with the primary signaling mediated by CD3.

The co-stimulatory domain(s) are located between the transmembrane domain and the CD3 signaling domain. Table 3 includes examples of suitable co-stimulatory domains together with the sequence of the CD35 signaling domain.

TABLE 3
CD3ζ Domain and Examples of Co-stimulatory Domains

| Name | Accession | Length | Sequence |
|---|---|---|---|
| CD3ζ | J04132.1 | 113 aa | RVKFSRSADAPAYQQGQNQLYNELNLGRREEYD |
| | | | VLDKRRGRDPEMGGKPRRKNPQEGLYNELQKDK |
| | | | MAEAYSEIGMKGERRRGKGHDGLYQGLSTATKD |
| | | | TYDALHMQALPPR (SEQ ID NO: 35) |
| | | | ITAMS 1-3 underlined |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAFSEIGMKGERRRGKGHDGLFQGLSTATKD |
| | | | TFDALHMQALPPR (SEQ ID NO: 50) |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAFSEIGMKGERRRGKGHDGLYQGLSTATKD |
| | | | TYDALHMQALPPR (SEQ ID NO: 51) |
| CD3ζ | | 113 aa | RVKFSRSADAPAYQQGQNQLYNELNLGRREEYD |
| variant | | | VLDKRRGRDPEMGGKPRRKNPQEGLYNELQKDK |
| | | | MAEAYSEIGMKGERRRGKGHDGLFQGLSTATKD |
| | | | TFDALHMQALPPR (SEQ ID NO: 52) |
| CD3ζ | | 113 aa | RVKFSRSADAPAYQQGQNQLYNELNLGRREEYD |
| variant | | | VLDKRRGRDPEMGGKPRRKNPQEGLYNELQKDK |
| | | | MAEAFSEIGMKGERRRGKGHDGLFQGLSTATKD |
| | | | TFDALHMQALPPR (SEQ ID NO: 53) |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAYSEIGMKGERRRGKGHDGLFQGLSTATKD |
| | | | TFDALHMQALPPR (SEQ ID NO: 54) |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAFSEIGMKGERRRGKGHDGLYQGLSTATKD |
| | | | TFDALHMQALPPR (SEQ ID NO: 55) |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAFSEIGMKGERRRGKGHDGLFQGLSTATKD |
| | | | TYDALHMQALPPR (SEQ ID NO: 56) |
| CD28 | NM_006139 | 42 aa | RSKRSRLLHSDYMNMTPRRPGPTRKHYQPYAPPR |
| | | | DFAAYRS (SEQ ID NO: 36) |
| CD28gg* | NM_006139 | 42 aa | RSKRSRGGHSDYMNMTPRRPGPTRKHYQPYAPPR |
| | | | DFAAYRS (SEQ ID NO: 37) |
| 41BB | NM_001561 | 42 aa | KRGRKKLLYIFKQPFMRPVQTTQEEDGCSCRFPEE |
| | | | EEGGCEL (SEQ ID NO: 38) |
| OX40 | NM_003327 | 42 aa | ALYLLRRDQRLPPDAHKPPGGGSFRTPIQEEQADA |
| | | | HSTLAKI (SEQ ID NO:39) |
| 2B4 | NM_016382 | 120 aa | WRRKRKEKQSETSPKEFLTIYEDVKDLKTRRNHE |
| | | | QEQTFPGGGSTIYSMIQSQSSAPTSQEPAYTLYSLI |
| | | | QPSRKSGSRKRNHSPSFNSTIYEVIGKSQPKAQNP |
| | | | ARLSRKELENFDVYS (SEQ ID NO: 40) |

In some examples, the CD32 signaling domain comprises an amino acid sequence that is at least 90%, at least 95%, at least 98% identical to SEQ ID NO: 35. In such instances, the CD3ζ signaling domain has 1, 2, 3, 4, or 5 amino acid changes (preferably conservative substitutions) compared to SEQ ID NO: 35. In other examples, the CD35 signaling domain is SEQ ID NO: 35.

In various embodiments: the co-stimulatory domain is selected from the group consisting of: a co-stimulatory domain depicted in Table 3 or a variant thereof having 1-5 (e.g., 1 or 2) amino acid modifications, a CD28 co-stimulatory domain or a variant thereof having 1-5 (e.g., 1 or 2) amino acid modifications, a 4-1BB co-stimulatory domain or a variant thereof having 1-5 (e.g., 1 or 2) amino acid modifications and an OX40 co-stimulatory domain or a variant thereof having 1-5 (e.g., 1 or 2) amino acid modifications. In certain embodiments, a 4-1BB co-stimulatory domain or a variant thereof having 1-5 (e.g., 1 or 2) amino acid modifications is present in the CAR polypeptides described herein.

In some embodiments, there are two co-stimulatory domains, for example, a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. In various embodiments, the co-stimulatory domain is amino terminal to the CD3 signaling domain and a short linker consisting of 2-10, e.g., 3 amino acids (e.g., GGG) is can be positioned between the co-stimulatory domain and the CD3ζ signaling domain.

II. Oncolytic HSVs and wtHSVs

Talimogene laherparepvec (T-VEC) is a genetically modified herpes simplex virus type 1 designed to selectively replicate in tumor cells. It is attenuated by the deletion of the genes, infectious cell protein (ICP) 34.5 and 47. Without being bound by theory, T-VEC combines direct oncolytic effects with local and systemic immune-mediated anti-tumoral effects, and the release of pro-inflammatory molecules, caused by the viral infection to activate the immune system. Any one or more of a variety of oncolytic HSV (oHSV), e.g., oncolytic HSV1, can be used in any of the methods disclosed herein. Various oHSV are described in Nguyen et al. (2021) Oncolytic Virology 10:1-27. Useful oHSV include:
T-VEC (lacks ICP34.5 and ICP47),
HF-10 (Takara Bio, Inc.; lacks UL43, UL49.5, UL55, UL56, and LAT),
HSV-1716 (Virttu Biologics; lacks ICP34.5),
G207 (Medigene; lacks ICP34.5 and ICP6 (substituted with LacZ)),
M032 (Acttis, Inc), and
G47Δ (Daiichi Sankyo Company; lacks ICP34.5, ICP6, and ICP47).

Others include: dlsptk (TK deleted), hrR3 (deltaICP6+LacZ), HSV1716 (−/−γ 34.5), HSV3616 (−/−γ 34.5), G207 (−/−γ34.5, ΔICP6, +LacZ), G47Delta (+/−γ34.5, ΔICP6, ΔICP47, +LacZ), rQNestin34.5v.2 (−γ34.5, ΔICP6, γ34.5 driven by nestin enhancer/promoter, +EGFP), MG18L (−US3, ΔICP6, +LacZ). Other also include Replimune (RP1, RP2, RP3) natural isolates engineered to carry transgenes; Oncorus (ONCR-177, ONCR-GBM, ONCR-021, ONCR-788); Peking Union Medical College (OH2) vOV designed from HSV-2.

Any one or more of a variety of wild-type HSV (wtHSV), e.g., HSV1 and HSV2, can be used in any of the methods disclosed herein.

Viruses being developed as vaccines for HSV1 (cold sores) or HSV2 (genital herpes) can also be used in any of the methods disclosed herein. This includes HSV529 (Sanolfi Pasteur; UL5 and UL29 defective virus).

### III. Preparation of Anti-gD CAR T Cells

In some cases, the gD CAR can be produced using a vector in which the CAR open reading frame is followed by a T2A ribosome skip sequence and a truncated EGFR (EGFRt), which lacks the cytoplasmic signaling tail, or a truncated CD19R (also called CD19t). In this arrangement, co-expression of EGFRt or CD19t provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt or the CD19t incorporated in the gD CAR lentiviral vector can act as suicide gene to ablate the CAR+ T cells in cases of treatment-related toxicity.

The CD35ζ signaling domain can be followed by a ribosomal skip sequence (e.g., LEGGGEGRGSLLTCGDVEENPGPR; SEQ ID NO: 45) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO: 46). In some cases, the truncated EGFR has 1, 2, 3, 4 of 5 amino acid changes (preferably conservative) compared to SEQ ID NO: 46.

Alternatively the CD3ζ signaling domain can be followed by a ribosomal skip sequence (e.g., LEGGGEGRGSLLTCGDVEENPGPR; SEQ ID NO: 45) and a truncated CD19R (also called CD19t) having a sequence that is at least 90%, at least 95%, at least 98% identical to or identical to:

(SEQ ID NO: 47)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCVPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKR.

Any CAR or polypeptide described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region can be inserted into an expression vector and used to transform a suitable expression host cell line. A suitable host cell line includes, for example, a T lymphocyte (including an autologous T lymphocyte), an NK cell, etc. An expression vector encoding a CAR or polypeptide described herein can be a viral vector. Suitable viral vectors, including lentiviral vectors, are known in the art and can be used in any of the methods described herein. In some aspects, any of the transduced immune cells described herein can be autologous or allogenic. For example, suitable cell populations can include allogenic NK cells, autologous NK cells, allogenic T cells, autologous T cells that harbor a nucleic acid encoding any CAR or polypeptide described herein. Suitable cell populations can also include allogenic NK cells, autogenic NK cells, allogenic T cells, autogenic T cells express any CAR or polypeptide described here.

Various T cell subsets isolated from the patient can be transduced with a vector for CAR or polypeptide expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an anti-gD CAR or as well as a non-immunogenic surface marker for in vivo detection, ablation, and potential ex vivo selection. The activated/genetically modified central memory T cells can be expanded in vitro with IL-2/IL-15 and then cryopreserved. Additional methods of preparing CAR T cells can be found in PCT/US2016/043392.

Methods for preparing useful T cell populations are described in, for example, WO 2017/015490 and WO 2018/102761. In some cases, it may be useful to use natural killer (NK) cells, e.g., allogenic NK cells derived from peripheral blood or cord blood. In other cases, NK cells can be derived from human embryonic stem cells (hESCs) or induced pluripotent stem cells (iPSCs).

In some embodiments, described herein is a composition comprising the iPSC-derived CAR T cells or CAR NK cells. In some embodiments, a composition comprising iPSC-derived CAR T cells or CAR NK cells has enhanced therapeutic properties. In some embodiments, the iPSC-derived CAR T cells or CAR NK cells demonstrate enhanced functional activity including potent cytokine production, cytotoxicity and cytostatic inhibition of tumor growth, e.g., as activity that reduces the amount of tumor load.

The CAR can be transiently expressed in a T cell population by an mRNA encoding the CAR. The mRNA can be introduced into the T cells by electroporation (Wiesinger et al. 2019 Cancers (Basel) 11:1198).

In some embodiments, a composition comprising the CAR T cells comprise one or more of helper T cells, cytotoxic T cells, memory T cells, naïve T cells, regulatory T cells, natural killer T cells, or combinations thereof.

In some examples, the anti-gD CAR comprises an amino acid sequence that is at least 90%, at least 95%, at least 98% identical to or identical to:

MLLLVTSLLLCELPHPAFLLIPQVTLKQSGAEVKKPGSSVKVSCTASGGT
LRTYGVSWVRQAPGQGLEWLGRTIPLFGKTDYAQKFQGRVTITADKSMDT
SFMELTSLTSEDTAVYYCARDLTTLTSYNWWDLWGQGTLVTVSSGGGGSG
GGGSGGGGSEIVLTQSPGTLSLSPGERATLSCRASQSVTSSQLAWYQQKP
GQAPRLLISGASNRATGIPDRFSGSGSGTDFTLTISRLEPEDFAVYYCQQ
YGSSPTFGGGTKVEIKRESKYGPPCPPCPGGGSSGGGSGGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKFWVL
VVVGGVLACYSLLVTVAFIIFWVKRGRKKLLYIFKQPFMRPVQTTQEEDG
CSCRFPEEEEGGCELGGGRVKFSRSADAPAYQQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR
(SEQ ID NO: 48; Pf04022; HSVscFv(gD, E317)-IgG4
(HL-CH3)-CD28tm-41BB-Zeta with signal sequence)
or
QVTLKQSGAEVKKPGSSVKVSCTASGGTLRTYGVSWVRQAPGQGLEWLGR
TIPLFGKTDYAQKFQGRVTITADKSMDTSFMELTSLTSEDTAVYYCARDL
TTLTSYNWWDLWGQGTLVTVSSGGGGSGGGGSGGGGSEIVLTQSPGTLSL
SPGERATLSCRASQSVTSSQLAWYQQKPGQAPRLLISGASNRATGIPDRF
SGSGSGTDFTLTISRLEPEDFAVYYCQQYGSSPTFGGGTKVEIKRESKYG
PPCPPCPGGGSSGGGSGGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSLGKFWVLVVVGGVLACYSLLVTVAFIIFW
VKRGRKKLLYIFKQPFMRPVQTTQEEDGCSCRFPEEEEGGCELGGGRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPR
(SEQ ID NO: 49; Pf04022; HSVscFv(gD, E317)-IgG4
(HL-CH3)-CD28tm-41BB-Zeta without signal sequence)

In some examples, the anti-gD CAR comprises an amino acid sequence that is at least 90%, at least 95%, at least 98% identical to or identical to:

MLLLVTSLLLCELPHPAFLLIPQVTLKQSGAEVKKPGSSVKVSCTASGGT
LRTYGVSWVRQAPGQGLEWLGRTIPLFGKTDYAQKFQGRVTITADKSMDT
SFMELTSLTSEDTAVYYCARDLTTLTSYNWWDLWGQGTLVTVSSGGGGSG
GGGSGGGGSEIVLTQSPGTLSLSPGERATLSCRASQSVTSSQLAWYQQKP
GQAPRLLISGASNRATGIPDRFSGSGSGTDFTLTISRLEPEDFAVYYCQQ
YGSSPTFGGGTKVEIKRESKYGPPCPPCPGGGSSGGGSGGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKFWVL
VVVGGVLACYSLLVTVAFIIFWVRSKRSRGGHSDYMNMTPRRPGPTRKHY
QPYAPPRDFAAYRSGGGRVKFSRSADAPAYQQGQNQLYNELNLGRREEYD
VLDKRRGRDPEMGGKPRRKNPQEGLYNELQKDKMAEAYSEIGMKGERRRG
KGHDGLYQGLSTATKDTYDALHMQALPPR
(SEQ ID NO: 57; Pf04023; HSVscFv(gD, E317)-IgG4
(HL-CH3)-CD28tm-CD28gg-Zeta with signal sequence)
or
QVTLKQSGAEVKKPGSSVKVSCTASGGTLRTYGVSWVRQAPGQGLEWLGR
TIPLFGKTDYAQKFQGRVTITADKSMDTSFMELTSLTSEDTAVYYCARDL
TTLTSYNWWDLWGQGTLVTVSSGGGGSGGGGSGGGGSEIVLTQSPGTLSL
SPGERATLSCRASQSVTSSQLAWYQQKPGQAPRLLISGASNRATGIPDRF
SGSGSGTDFTLTISRLEPEDFAVYYCQQYGSSPTFGGGTKVEIKRESKYG
PPCPPCPGGGSSGGGSGGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSLGKFWVLVVVGGVLACYSLLVTVAFIIFW
VRSKRSRGGHSDYMNMTPRRPGPTRKHYQPYAPPRDFAAYRSGGGRVKFS
RSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALH
MQALPPR
(SEQ ID NO: 58; Pf04023; HSVscFv(gD, E317)-IgG4
(HL-CH3)-CD28tm-CD28gg-Zeta without signal
sequence)

Disclosed herein, amongst other things, are methods of making any CAR or polypeptide described herein. Disclosed herein, amongst other things, are methods of making a population of T cells and/or NK cells comprising a nucleic acid encoding any CAR or polypeptide described herein. Disclosed herein, amongst other things, are methods of making a population of T cells and/or NK cells expressing any CAR or polypeptide described herein.

IV. Treatment of Cancer and/or HSV

Aspects of the present disclosure provide methods for treating a subject having cells expressing gD (e.g., cells infected by HSV1 or HSV2) and/or a subject having a cancer wherein the cells can be made to express gD, for example, by infecting cancer cells with an oHSV or an wtHSV.

### (a) Subjects

Subjects in need of treatment can have cells expressing gD and/or have an active HSV1 and/or HSV2 infection and/or have cancer cells that can be induced to express gD.

A subject to be treated by the methods described can be a human patient having a cancer, such as a solid tumor, e.g., gastrointestinal cancer, breast cancer, lung cancer, bladder cancer, thyroid cancer, and ovarian cancer. Non-limiting examples of gastrointestinal cancers include colon cancer, gastric cancer, rectal cancer, pancreatic cancer, and combinations thereof.

A subject at risk of having cancer might show one or more symptoms of a gD-expressing cancer, e.g., unexplained weight loss, fatigue, pain, persistent cough, lumps under the skin, or unusual bleeding. A subject at risk of having cancer might have one or more risk factors of a cancer, e.g., family history of cancer, age, tobacco use, obesity, or exposure to sun or carcinogens. A subject who needs the treatment described herein can be identified by routine medical examination, e.g., laboratory tests, biopsy, magnetic resonance imaging (MRI), or ultrasound exams.

A subject having HSV1 and/or HSV2 might show one or more of the symptoms of a cold sore, a genital wart, pain or sensitivity in the area at or around the lips, pain or sensitivity in the area at or around the genitals, fever, nausea, headaches, muscle aches, painful urination, and vaginal discharge.

### (b) Treatment Regimens

Aspects of the present disclosure provide methods of treating a solid tumor comprising administering a lymphodepletion treatment (e.g., cyclophosphamide) in combination with gD CAR immune cells and an oHSV or a wtHSV, each of which can be administered locally or systemically. The two components can be administered the same day or on different days. The administration of gD CAR immune cells should be timed such that cells infected by oHSV or wtHSV have an opportunity to express cell surface gD.

Aspects of the present disclosure also provide methods of treating a cancer comprising administering to a subject having a cancer a population of gD CAR immune cells (e.g., gD-CAR T cells and/or gD-CAR NK cells) and an oHSV or a wtHSV, each of which can be administered locally or systemically.

Aspects of the present disclosure also provide methods of treating HSV-1 and/or HSV-2 comprising administering to a subject having HSV-1 and/or HSV-2 a population of gD CAR immune cells (e.g., gD-CAR T cells and/or gD-CAR NK cells), which can be administered locally or systemically. A population of gD CAR immune cells can be administered in a single dose or in repeat dosing. During repeat dosing, each dose of the anti-gD CAR immune cells can be the same or the doses can increase or decrease.

Generally, the methods include administering a therapeutically effective amount of a population of gD-CAR T cells and/or gD-CAR NK cells as described herein, to a subject who is in need of, or who has been determined to be in need of, such treatment.

The population of gD CAR immune cells in all compositions and methods disclosed herein can be autologous or allogenic.

Any subject suitable for the treatment methods described herein can receive a lymphodepleting therapy to reduce or deplete the endogenous lymphocytes of the subject. Lymphodepletion refers to the destruction of endogenous lymphocytes and/or T cells, which is commonly used prior to immunotransplantation and immunotherapy. Lymphodepletion can be achieved by administering a lymphodepleting agent and/or irradiation (e.g., stereotactic radiation). A lymphodepleting agent can be any molecule capable of reducing, depleting, or eliminating endogenous lymphocytes and/or T cells when administered to a subject. In some examples, the lymphodepleting agents are cytotoxic agents that specifically kill lymphocytes. Non-limiting examples of lymphodepleting agents include cyclophosphamide, fludarabine, gemcitabine, methotrexate, doxorubicin, and etopside phosphate. In some cases the lymphodepletion treatment is non-myeloablative.

Methods described herein can include a conditioning regimen comprising a single lymphodepleting agent (e.g., cyclophosphamide) or multiple lymphodepleting agents (e.g., cyclophosphamide and fludarabine). The subject to be treated by the methods described herein can receive one or more doses of the one or more lymphodepleting agents for a period suitable for reducing or depleting the endogenous lymphocytes of the subject (e.g., 1-5 days).

The subject can then be administered any of the anti-gD CAR immune cells described herein after administration of the lymphodepleting therapy as described herein. For example, the one or more lymphodepleting agents can be administered to the subject 1-5 days (e.g., 1, 2, 3, 4, or 5 days) prior to administering the anti-gD CAR T cells.

Methods described herein can include redosing the subject with anti-gD CAR immune cells. In some examples, the subject is administered a lymphodepleting treatment prior to redosing of the anti-CAR immune cells. Each dose of the anti-gD CAR immune cells can be the same or the doses can be ascending or descending.

The oHSV can be administered to the subject 1-5 days (e.g., 1, 2, 3, 4, or 5 days) after administering the anti-gD CAR immune cells.

Methods described herein can include redosing the subject with gD CAR immune cells. In some examples, the subject is administered 3-6 doses of the gD CAR immune cells, each of which is administered 1-15 days after the prior dose. Each dose of gD CAR immune cells can be the same or the doses can be ascending or descending.

In each case, the gD CAR immune cells can be T cells or NK cells.

Methods described herein can be used in combination with another anti-cancer therapy (e.g., chemotherapy), with another anti-viral therapy (e.g., Famvir (famciclovir); Valtrex (valacyclovir); Zovirax (acyclovir); Abreva), or with another therapeutic agent that reduces side effects of the therapy described herein.

### (c) Administration

An effective amount of a therapy (e.g., lymphodepleting agent, anti-gD CAR T cells, oHSV) can be administered to a subject (e.g., a human) in need of the treatment via any suitable route (e.g., administered locally or systemically to a subject). Suitable modes of administration include injection, infusion, instillation, or ingestion. Injection includes, without limitation, intravenous, intramuscular, intra-arterial, intrathecal, intraventricular, intradermal, intraperitoneal, and subcutaneous injection and infusion.

An effective amount, or therapeutically effective amount, refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of treatment, the nature of concurrent therapy, if any, the specific route of administration and like factors within the knowledge and expertise of the health practitioner. The amelioration of one symptom associated with the condition, cancer, or disease is enough to confer therapeutic effect on the subject. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Disclosed herein, amongst other things, methods of administering to a subject in need thereof (e.g., a subject having HSV), a therapeutic amount of any disclosed cell population comprising a nucleic acid encoding any CAR or polypeptide described herein. Disclosed herein, amongst other things, methods of administering to a subject in need thereof (e.g., a subject having HSV), a therapeutic amount of any disclosed cell population expressing any CAR or polypeptide described herein.

The materials, methods, and examples are illustrative only and not intended to be limiting. All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### BRIEF DESCRIPTION OF THE DRAWINGS

FIG. 1: Quantification of gD expression (left) and viability (right) of MDA-MB-468 tumor cells after 24-, 48-, and 72-hours exposure to the indicated MOIs of HSV.

FIG. 2: Images showing U251T glioma cells (U251T) or U251T glioma cells stably infected with a lentivirus expressing gD (U251-gD) exposed to mock T cells, Pf04023 gD CAR T cells, or HSV treatment (MOI of 0.01) in combination with either mock T cells or Pf04023 gD CAR T cells.

FIG. 3: Quantification of MDA-MB-468 tumor cell killing assessed by flow cytometry. MDA-MB-468 tumor cells were co-cultured with untransduced T cells (Mock) or gD-CAR T cells for 24, 48, and 72 hours in the presence of indicated MOIs of HSV. Values for MDA-MB-468 cells stably expressing glycoprotein-D co-cultured with gD-CAR T cells are indicated by a single data point on each graph (dot indicated by arrow).

FIG. 4: Percent of MDA-MB-468 cells positive for glycoprotein-D in killing assay following HSV alone or in combination with gD-CAR T cells at indicated timepoints.

FIG. 5: IFNγ production measured by enzyme-linked immunosorbent assay (ELISA) in supernatants collected from co-cultures of MDA-MB-468 tumor cells alone, with mock (untransduced) T cells, or with gD-CAR T cells in the presence or absence of HSV at indicated MOIs for 24, 48, and 72 hours.

FIG. 6: Quantification of CD137 (left) and CD69 (right) expression on Mock or gD-CAR T cells following 24-hour co-culture with MDA-MB-468 tumor cells with or without indicated MOIs of HSV.

FIG. 7: Quantification of viable cells (left) and gD expression (right) of MDA-MB-468 tumor cells after 24 hours of exposure to the indicated MOIs of HSV, T-VEC (stored at 4° C. without freeze thaw), and T-VEC (stored at −80° C. with one freeze-thaw cycle) assessed by flow cytometry.

FIG. 8: Quantification of gD expression (left) and viability (right) of MDA-MB-468 tumor cells after 24-, 48-, and 72-hours exposure to the indicated MOIs of T-VEC.

FIG. 9: Quantification of MDA-MB-468 tumor cell killing assessed by flow cytometry. MDA-MB-468 tumor cells were co-cultured with untransduced T cells (Mock) or gD-CAR T cells for 24, 48, and 72 hours in the presence of indicated MOIs of T-VEC or T-VEC alone also at the indicated MOIs. Values for MDA-MB-468 cells stably expressing glycoprotein-D co-cultured with gD-CAR T cells are indicated by a single data point on each graph (blue dot).

FIG. 10: Percent of MDA-MB-468 cells positive for glycoprotein-D in killing assay following T-VEC alone or in combination with gD-CAR T cells or in combination with untransduced T cells (Mock) after 24, 48, and 72 hours (far left graph, middle graph, and right graph, respectively).

FIG. 11: Quantification of CD137 on Mock or gD-CAR T cells following 24, 48, and 72 hours co-culture with MDA-MB-468 tumor cells with or without indicated MOIs of T-VEC.

FIG. 12: Depicts the amino acid sequence of HSVscFv(gD,E317)-IgG4(HL-CH3)-CD28tm-41BB-Zeta, including the signal sequence. The various domains are indicated. The amino acid sequence including the signal sequence is SEQ ID NO: 48; without the signal sequence, SEQ ID NO: 49.

FIG. 13: Depicts the amino acid sequence of HSVscFv (gD,E317)-IgG4 (HL-CH3)-CD28tm-CD28gg-Zeta, including the signal sequence. The various domains are indicated. The amino acid sequence including the signal sequence is SEQ ID NO: 57; without the signal sequence, SEQ ID NO: 58.

FIG. 14: Depicts the amino acid sequence of HSVscFv (gD,E317)-IgG4 (HL-CH3)-CD28tm-CD28gg-41BB-Zeta, including the signal sequence. The various domains are indicated. The amino acid sequence including the signal sequence is SEQ ID NO: 59; without the signal sequence, SEQ ID NO: 60.

FIG. 15: Depicts the amino acid sequence of HSVscFv (gD,E317)-IgG4 (HL-CH3)-CD28 (M) tm-41BB-Zeta, including the signal sequence. The various domains are indicated. The amino acid sequence including the signal sequence is SEQ ID NO: 61; without the signal sequence, SEQ ID NO: 62.

FIG. 16: Depicts the amino acid sequence of HSVscFv (gD,E317)-IgG4 (HL-CH3)-CD28 (M) tm-CD28gg-Zeta, including the signal sequence. The various domains are indicated. The amino acid sequence including the signal sequence is SEQ ID NO: 63; without the signal sequence, SEQ ID NO: 64.

FIG. 17: Depicts the amino acid sequence of HSVscFv (gD,E317)-IgG4 (HL-CH3)-CD28 (M) tm-CD28gg-41BB-Zeta, including the signal sequence. The various domains are indicated. The amino acid sequence including the signal sequence is SEQ ID NO: 65; without the signal sequence, SEQ ID NO: 66.

FIG. 18: Depicts the amino acid sequence of HSVscFv (gD,E317)-IgG4 (HL-CH3)-CD4tm-41BB-Zeta, including the signal sequence. The various domains are indicated. The amino acid sequence including the signal sequence is SEQ ID NO: 67; without the signal sequence, SEQ ID NO: 68.

FIG. 19: Depicts the amino acid sequence of HSVscFv (gD,E317)-IgG4 (HL-CH3)-CD4tm-CD28gg-Zeta, including the signal sequence. The various domains are indicated. The amino acid sequence including the signal sequence is SEQ ID NO: 69; without the signal sequence, SEQ ID NO: 70.

FIG. 20: Depicts the amino acid sequence of HSVscFv (gD,E317)-IgG4 (HL-CH3)-CD4tm-CD28gg-41BB-Zeta, including the signal sequence. The various domains are indicated. The amino acid sequence including the signal sequence is SEQ ID NO: 71; without the signal sequence, SEQ ID NO: 72.

FIG. 21: Depicts the amino acid sequence of HSVscFv (gD,E317)-IgG4 (HL-CH3)-CD8tm-41BB-Zeta, including the signal sequence. The various domains are indicated. The amino acid sequence including the signal sequence is SEQ ID NO: 73; without the signal sequence, SEQ ID NO: 74.

FIG. 22: Depicts the amino acid sequence of HSVscFv (gD,E317)-IgG4 (HL-CH3)-CD8tm-CD28gg-Zeta, including the signal sequence. The various domains are indicated. The amino acid sequence including the signal sequence is SEQ ID NO: 75; without the signal sequence, SEQ ID NO: 76.

FIG. 23: Depicts the amino acid sequence of HSVscFv (gD,E317)-IgG4 (HL-CH3)-CD8tm-CD28gg-41BB-Zeta, including the signal sequence. The various domains are indicated. The amino acid sequence including the signal sequence is SEQ ID NO: 77; without the signal sequence, SEQ ID NO: 78.

FIG. 24: Depicts the amino acid sequence of HSVscFv (gD,E317)-IgG4 (S228P, L235E,N297Q)-CD28tm-41BB-Zeta, including the signal sequence. The various domains are indicated. The amino acid sequence including the signal sequence is SEQ ID NO: 79; without the signal sequence, SEQ ID NO: 80.

FIG. 25: Depicts the amino acid sequence of HSVscFv (gD,E317)-IgG4 (S228P, L235E,N297Q)-CD28tm-CD28gg-Zeta, including the signal sequence. The various domains are indicated. The amino acid sequence including the signal sequence is SEQ ID NO: 81; without the signal sequence, SEQ ID NO: 82.

FIG. 26: Depicts the amino acid sequence of HSVscFv (gD,E317)-IgG4 (S228P, L235E,N297Q)-CD28tm-CD28gg-41BB-Zeta, including the signal sequence. The various domains are indicated. The amino acid sequence including the signal sequence is SEQ ID NO: 83; without the signal sequence, SEQ ID NO: 84.

FIG. 27: Depicts the amino acid sequence of HSVscFv (gD,E317)-IgG4 (S228P, L235E,N297Q)-CD28 (M) tm-41BB-Zeta, including the signal sequence. The various domains are indicated. The amino acid sequence including the signal sequence is SEQ ID NO: 85; without the signal sequence, SEQ ID NO: 86.

FIG. 28: Depicts the amino acid sequence of HSVscFv (gD,E317)-IgG4 (S228P, L235E,N297Q)-CD28 (M) tm-CD28gg-Zeta, including the signal sequence. The various domains are indicated. The amino acid sequence including the signal sequence is SEQ ID NO: 87; without the signal sequence, SEQ ID NO: 88.

FIG. 29: Depicts the amino acid sequence of HSVscFv (gD,E317)-IgG4 (S228P, L235E,N297Q)-CD28 (M) tm-CD28gg-41BB-Zeta, including the signal sequence. The various domains are indicated. The amino acid sequence including the signal sequence is SEQ ID NO: 89; without the signal sequence, SEQ ID NO: 90.

FIG. 30: Depicts the amino acid sequence of HSVscFv (gD,E317)-IgG4 (S228P, L235E,N297Q)-CD4tm-41BB-Zeta, including the signal sequence. The various domains are indicated. The amino acid sequence including the signal sequence is SEQ ID NO: 91; without the signal sequence, SEQ ID NO: 92.

FIG. 31: Depicts the amino acid sequence of HSVscFv (gD,E317)-IgG4 (S228P, L235E,N297Q)-CD4tm-CD28gg-Zeta, including the signal sequence. The various domains are indicated. The amino acid sequence including the signal sequence is SEQ ID NO: 93; without the signal sequence, SEQ ID NO: 94.

FIG. 32: Depicts the amino acid sequence of HSVscFv (gD,E317)-IgG4 (S228P, L235E,N297Q)-CD4tm-CD28gg-41BB-Zeta, including the signal sequence. The various domains are indicated. The amino acid sequence including the signal sequence is SEQ ID NO: 95; without the signal sequence, SEQ ID NO: 96.

FIG. 33: Depicts the amino acid sequence of HSVscFv (gD,E317)-IgG4 (S228P, L235E,N297Q)-CD8tm-41BB-Zeta, including the signal sequence. The various domains are indicated. The amino acid sequence including the signal sequence is SEQ ID NO: 97; without the signal sequence, SEQ ID NO: 98.

FIG. 34: Depicts the amino acid sequence of HSVscFv (gD,E317)-IgG4 (S228P, L235E,N297Q)-CD8tm-CD28gg-Zeta, including the signal sequence. The various domains are indicated. The amino acid sequence including the signal sequence is SEQ ID NO: 99; without the signal sequence, SEQ ID NO: 100.

FIG. 35: Depicts the amino acid sequence of HSVscFv (gD,E317)-IgG4 (S228P, L235E,N297Q)-CD8tm-CD28gg-41BB-Zeta, including the signal sequence. The various domains are indicated. The amino acid sequence including the signal sequence is SEQ ID NO: 101; without the signal sequence, SEQ ID NO: 102.

FIG. 36: Depicts the amino acid sequence of HSVscFv (gD,E317)-IgG4 (L235E,N297Q)-CD28tm-41BB-Zeta, including the signal sequence. The various domains are indicated. The amino acid sequence including the signal sequence is SEQ ID NO: 103; without the signal sequence, SEQ ID NO: 104.

FIG. 37: Depicts the amino acid sequence of HSVscFv (gD,E317)-IgG4 (L235E,N297Q)-CD28tm-CD28gg-Zeta, including the signal sequence. The various domains are indicated. The amino acid sequence including the signal sequence is SEQ ID NO: 105; without the signal sequence, SEQ ID NO: 106.

FIG. 38: Depicts the amino acid sequence of HSVscFv (gD,E317)-IgG4 (L235E,N297Q)-CD28tm-CD28gg-41BB-Zeta, including the signal sequence. The various domains are indicated. The amino acid sequence including the signal sequence is

SEQ ID NO: 107; without the signal sequence, SEQ ID NO: 108.

FIG. 39: Depicts the amino acid sequence of HSVscFv (gD,E317)-IgG4 (L235E,N297Q)-CD28 (M) tm-41BB-Zeta, including the signal sequence. The various domains are indicated. The amino acid sequence including the signal sequence is SEQ ID NO: 109; without the signal sequence, SEQ ID NO: 110.

FIG. 40: Depicts the amino acid sequence of HSVscFv (gD,E317)-IgG4 (L235E,N297Q)-CD28 (M) tm-CD28gg-Zeta, including the signal sequence. The various domains are indicated. The amino acid sequence including the signal sequence is SEQ ID NO: 111; without the signal sequence, SEQ ID NO: 112.

FIG. 41: Depicts the amino acid sequence of HSVscFv (gD,E317)-IgG4 (L235E,N297Q)-CD28 (M) tm-CD28gg-41BB-Zeta, including the signal sequence. The various domains are indicated. The amino acid sequence including the signal sequence is SEQ ID NO: 113; without the signal sequence, SEQ ID NO: 114.

FIG. 42: Depicts the amino acid sequence of HSVscFv (gD,E317)-IgG4 (L235E,N297Q)-CD4tm-41BB-Zeta, including the signal sequence. The various domains are indicated. The amino acid sequence including the signal sequence is SEQ ID NO: 115; without the signal sequence, SEQ ID NO: 116.

FIG. 43: Depicts the amino acid sequence of HSVscFv (gD,E317)-IgG4 (L235E,N297Q)-CD4tm-CD28gg-Zeta, including the signal sequence. The various domains are indicated. The amino acid sequence including the signal sequence is SEQ ID NO: 117; without the signal sequence, SEQ ID NO: 118.

FIG. 44: Depicts the amino acid sequence of HSVscFv (gD,E317)-IgG4 (L235E,N297Q)-CD4tm-CD28gg-41BB-Zeta, including the signal sequence. The various domains are indicated. The amino acid sequence including the signal sequence is SEQ ID NO: 119; without the signal sequence, SEQ ID NO: 120.

FIG. 45: Depicts the amino acid sequence of HSVscFv (gD,E317)-IgG4 (L235E,N297Q)-CD8tm-41BB-Zeta, including the signal sequence. The various domains are indicated. The amino acid sequence including the signal sequence is SEQ ID NO: 121; without the signal sequence, SEQ ID NO: 122.

FIG. 46: Depicts the amino acid sequence of HSVscFv (gD,E317)-IgG4 (L235E,N297Q)-CD8tm-CD28gg-Zeta, including the signal sequence. The various domains are indicated. The amino acid sequence including the signal sequence is SEQ ID NO: 123; without the signal sequence, SEQ ID NO: 124.

FIG. 47: Depicts the amino acid sequence of HSVscFv (gD,E317)-IgG4 (L235E,N297Q)-CD8tm-CD28gg-41BB-Zeta, including the signal sequence. The various domains are indicated. The amino acid sequence including the signal sequence is SEQ ID NO: 125; without the signal sequence, SEQ ID NO: 126.

FIG. 48: Depicts the amino acid sequence of HSVscFv (gD,E317)-L-CD28tm-41BB-Zeta, including the signal sequence. The various domains are indicated. The amino acid sequence including the signal sequence is SEQ ID NO: 103; without the signal sequence, SEQ ID NO: 104.

FIG. 49: Depicts the amino acid sequence of HSVscFv (gD,E317)-L-CD28tm-CD28gg-Zeta, including the signal sequence. The various domains are indicated. The amino acid sequence including the signal sequence is SEQ ID NO: 105; without the signal sequence, SEQ ID NO: 106.

FIG. 50: Depicts the amino acid sequence of HSVscFv (gD,E317)-L-CD28tm-CD28gg-41BB-Zeta, including the signal sequence. The various domains are indicated. The amino acid sequence including the signal sequence is SEQ ID NO: 107; without the signal sequence, SEQ ID NO: 108.

FIG. 51: Depicts the amino acid sequence of HSVscFv (gD,E317)-L-CD28 (M) tm-41BB-Zeta, including the signal sequence. The various domains are indicated. The amino acid sequence including the signal sequence is SEQ ID NO: 109; without the signal sequence, SEQ ID NO: 110.

FIG. 52: Depicts the amino acid sequence of HSVscFv (gD,E317)-L-CD28 (M) tm-CD28gg-Zeta, including the signal sequence. The various domains are indicated. The amino acid sequence including the signal sequence is SEQ ID NO: 111; without the signal sequence, SEQ ID NO: 112.

FIG. 53: Depicts the amino acid sequence of HSVscFv (gD,E317)-L-CD28 (M) tm-CD28gg-41BB-Zeta, including the signal sequence. The various domains are indicated. The amino acid sequence including the signal sequence is SEQ ID NO: 113; without the signal sequence, SEQ ID NO: 114.

FIG. 54: Depicts the amino acid sequence of HSVscFv (gD,E317)-L-CD4tm-41BB-Zeta, including the signal sequence. The various domains are indicated. The amino acid sequence including the signal sequence is SEQ ID NO: 115; without the signal sequence, SEQ ID NO: 116.

FIG. 55: Depicts the amino acid sequence of HSVscFv (gD,E317)-L-CD4tm-CD28gg-Zeta, including the signal sequence. The various domains are indicated. The amino acid sequence including the signal sequence is SEQ ID NO: 117; without the signal sequence, SEQ ID NO: 118.

FIG. 56: Depicts the amino acid sequence of HSVscFv (gD,E317)-L-CD4tm-CD28gg-41BB-Zeta, including the signal sequence. The various domains are indicated. The amino acid sequence including the signal sequence is SEQ ID NO: 119; without the signal sequence, SEQ ID NO: 120.

FIG. 57: Depicts the amino acid sequence of HSVscFv (gD,E317)-L-CD8tm-41BB-Zeta, including the signal sequence. The various domains are indicated. The amino acid sequence including the signal sequence is SEQ ID NO: 121; without the signal sequence, SEQ ID NO: 122.

FIG. 58: Depicts the amino acid sequence of HSVscFv (gD,E317)-L-CD8tm-CD28gg-Zeta, including the signal sequence. The various domains are indicated. The amino acid sequence including the signal sequence is SEQ ID NO: 123; without the signal sequence, SEQ ID NO: 124.

FIG. 59: Depicts the amino acid sequence of HSVscFv (gD,E317)-L-CD8tm-CD28gg-41BB-Zeta, including the signal sequence. The various domains are indicated. The amino acid sequence including the signal sequence is SEQ ID NO: 125; without the signal sequence, SEQ ID NO: 126.

FIGS. 60A-60B are bar graphs showing TVEC infected and induced surface expression of glycoprotein D onto solid mouse tumors in vitro. Flow cytometric analysis showed MOI dependent increase in percent gD (FIG. 60A) and PDL1 expression (FIG. 60B) on MC38 tumor cells following 24-, 48-, and 72-hour coculture with TVEC.

FIGS. 61A-61B are bar graphs that showed TVEC infected and induced surface expression of glycoprotein D onto solid mouse tumors in vitro. Flow cytometric analysis showed MOI dependent increase in percent gD (FIG. 61A) and PDL1 expression (FIG. 61B) on EMT6 tumor cells following 24-, 48-, and 72-hour coculture with TVEC.

FIGS. 62A-62B are FACS plots of murine gD-CAR T cells. (FIG. 62A) A FACS plot of gD-CAR (detected via mCD19 positivity) on the surface of ex vivo engineered enriched murine T cells infected with retrovirus carrying the gD-CAR construct. (FIG. 62B) A FACS plot of CD4+ and CD8+ population distributions of gD-mCAR expressing T cells.

FIGS. 63A-63F are plots that showed TVEC introduces gD on murine tumor cells, which directs cytotoxicity and activation of murine gD-CAR T cells in vitro. Quantification of mouse tumor cell killing assessed by flow cytometry. MC38 cells (FIG. 63A) and EMT6 cells (FIG. 63B) were cocultured with TVEC at the indicated MOIs with untransduced T cells (mock) or gD-mCAR T cells. Data presented are from duplicate wells from one experiment and shown as means+SEM. Percent of MC38 cells (FIG. 63C) and EMT6 cells (FIG. 63D) positive for gD and PDL1 in killing assay described in (FIGS. 63A-63B) assessed by flow cytometry at the indicated time points. Data presented are from duplicate wells from one experiment and shown as means+SEM. Activation of gD-mCAR T cells against TVEC-infected tumor cells expressing gD (MC38 cells (FIG. 63E) and EMT6 cells (FIG. 63F)). Quantification of T cell count and percent CD137 expression on untransduced T cells (mock) or gD-mCAR T cells in killing assay described in (FIGS. 63A-63B) assessed by flow cytometry at the indicated time points. Data presented are from duplicate wells from one experiment and shown as means+SEM.

FIGS. 64A-64E are plots that showed the antitumor efficacy of combination therapy of TVEC and murine gD-CAR T cells in an immunocompetent murine syngeneic tumor model. (FIG. 64A) C57BL/Bj mice were engrafted with subcutaneous (s.c.) MC38 tumors (5×10⁵ cells) and on day 8 were treated with intraperitoneal cyclophosphamide, and subsequently treated intratumorally (i.t.) with 5×107 plaque forming units (pfu) per mice per day on days 9 and 10. On day 11, mice were treated with murine gD-CAR T cells intratumorally. Tumor volumes were measured with calipers. Data for each mouse are shown for each group: mock only, FIG. 64B; gD-CAR only, FIG. 64C; TVEC+mock, FIG. 64D; and TVEC+gD CAR treatment, FIG. 64E.

FIG. 65 shows the plot of the Kaplan-Meier survival curves from the experiment described in (FIG. 64A).

The details of one or more embodiments of the invention are set forth in the description below. Other features or advantages of the present invention will be apparent from the drawings and detailed description of several embodiments, and also from the appended claims.

### DETAILED DESCRIPTION

In order that the invention described may be more fully understood, the following examples are set forth. The examples described in this application are offered to illustrate the methods and compositions provided herein and are not to be construed in any way as limiting their scope.

### Example 1: Preparation gD CAR T Cells

Two different gD CAR were generated. Both include a svFv (E317) that binds human HSV glycoprotein 1. In Pf04023, the scFv is followed by a modified IgG4 lacking the CH2 domain and including a linker (IgG4 (HL-ΔCH2); SEQ ID NO: 31), a CD28 transmembrane domain (SEQ ID NO: 16 or 17), a CD28gg co-stimulatory domain (SEQ ID NO: 37), a GGG spacer, a CD3 zeta domain (SEQ ID NO: 35). The CAR sequence is preceded by a signal sequence (SEQ ID NO:3) and followed by a T2A skip sequence (SEQ ID NO: 45) and a truncated CD19 sequence (lacking signaling function), allowing the gD CAR to be co-expressed with non-functional CD19 which can be used as a detectable marker.

In Pf04022, the scFv is followed by a modified IgG4 lacking the CH2 domain and including a linker (IgG4 (HL-ΔCH2; SEQ ID NO: 31), a CD28 transmembrane domain (SEQ ID NO: 16 or 17), a 41BB co-stimulatory domain (SEQ ID NO: 38), a GGG spacer, a CD3 zeta domain (SEQ ID NO: 35). The CAR sequence is preceded by a signal sequence (SEQ ID NO: 3) and followed by a T2A skip sequence (SEQ ID NO: 45) and a truncated CD19 sequence (lacking signaling function), allowing the gD CAR to be co-expressed with non-functional CD19 which can be used as a detectable marker.

### Example 2: Expression of gD on Tumor Cells Exposed to oHSV

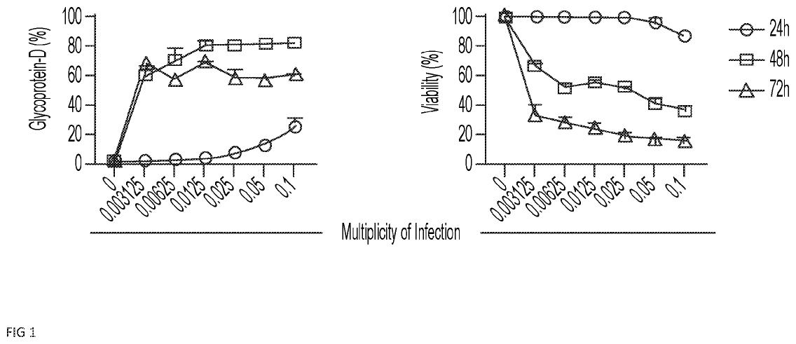

MDA-MB-468 human triple-negative breast cancer cells were exposed to an HSV at various MOI. Expression of gD and viability were measured after 24 hr, 48 hr, and 72 hr after exposure to virus. As can be seen in FIG. 1, HSV elicited gD expression and reduced viability of the cancer cells.

### Example 3: gD CAR T Cells Enhance Tumor Cell Killing by HSV

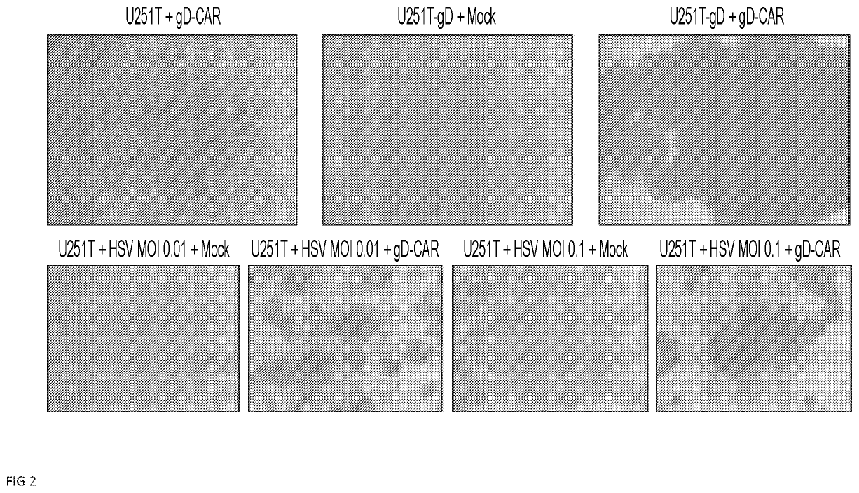

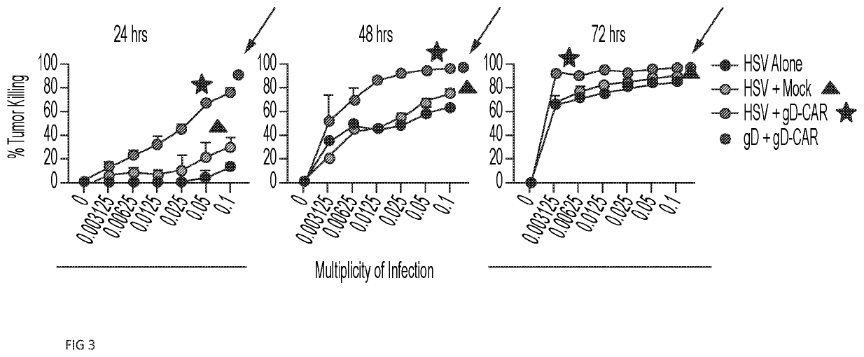

U251T glioma cells (U251T) or U251T glioma cells stably infected with a lentivirus expressing gD (U251-gD) were exposed to Pf04023 gD CAR T, which killed the stably transfected cells, but did not kill the non-transfected cells (FIG. 3). U251 T cells were exposed to Pf04023 gD CAR T, Pf04023 gD CAR T or HSV (MOI of 0.01) in combination with Pf04023 gD CAR T cells. As can be seen in FIG. 2, the combination was effective in killing tumor cells.

MDA-MB-468 cells were co-cultured with untransduced T cells (mock) or gd CAR T cells for 24, 48 or 72 hours in the presence of HSV at various MOI and cell viability was measured. As can be seen in FIG. 3, gD CAR T cells enhanced tumor cell killing by HSV.

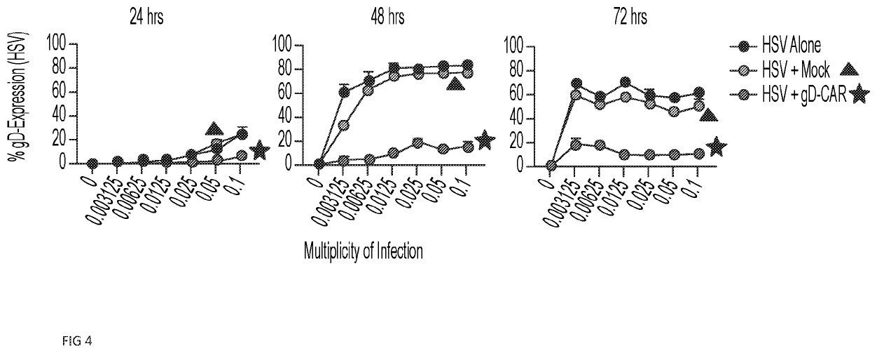

MDA-MB-468 cells were co-cultured with untransduced T cells (mock) or gd CAR T cells for 24, 48 or 72 hours in the presence of HSV at various MOI and the percent of HSV infected cells that express gD was measured. As can be seen in FIG. 4, gD CAR T cells were specifically targeting HSV infected tumor cells expressing gD.

### Example 4: Cytokine Expression of gD CAR T Cells

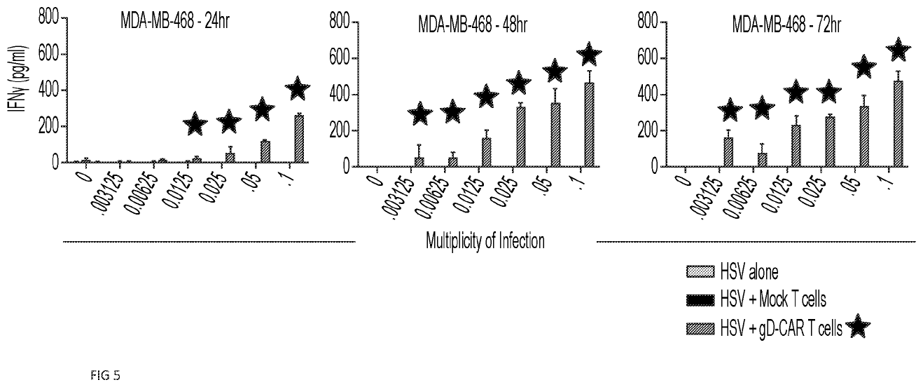

IFNγ production measured by enzyme-linked immunosorbent assay (ELISA) in supernatants collected from co-cultures of MDA-MB-468 tumor cells alone, with mock (untransduced) T cells, or with gD-CAR T cells in the presence or absence of HSV at various MOIs for 24, 48, and 72 hours. As can be seen in FIG. 5, gD CAR T cells exposed to tumor cells exposed to HSV express IFNγ.

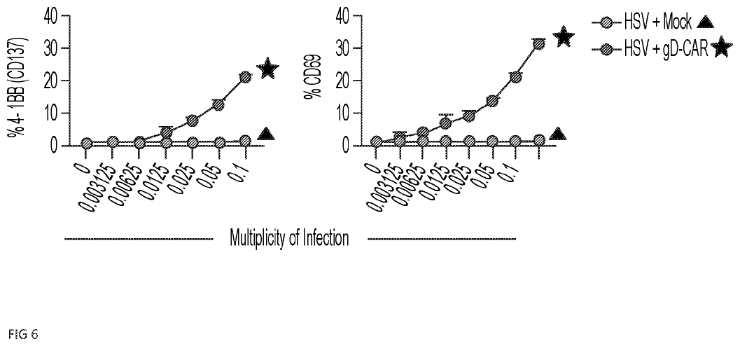

CD137 and CD69 expression by mock transfected T cells and gD CAR T cells was measured following 24-hour co-culture with MDA-MB-468 tumor cells and MDA-MB-468 tumor cells exposed to HSV at various MOI. As can be seen in FIG. 6, gD CAR T cells exposed to tumor cells cultured with HSV express CD137 and CD69.

### Example 5: gD CAR T Cells Enhance Tumor Cell Killing by HSV

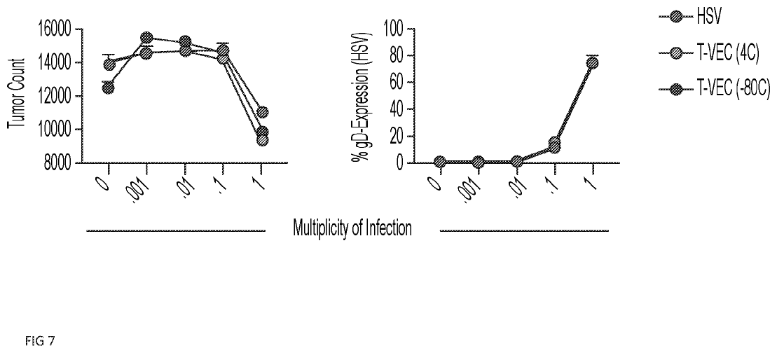

MDA-MB-468 tumor cells were co-cultured with HSV or Talimogene laherparepvec (T-VEC) at various MOI. Tumor cell count and the percent of HSV infected cells expressing gD was measured. As can be seen in FIG. 7, the results with HSV and T-VEC were comparable.

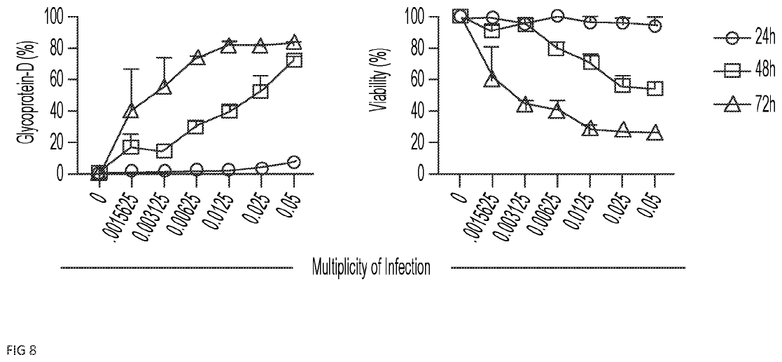

MDA-MB-468 tumor cells were cultured with T-VEC at various MO1 for 24, 48 or 72 hours. Percent of gD expressing cells and cell viability was measured. As can be seen in FIG. 8, both gD expression and viability were time and MOI dependent when MDA-MB-468 tumor cells were infected with T-VEC.

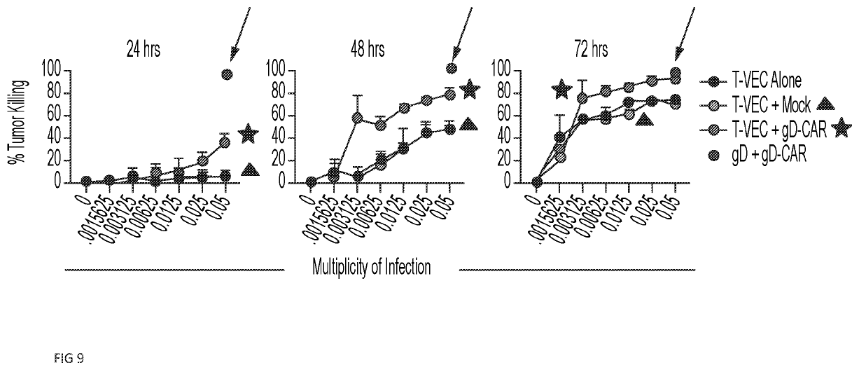

MDA-MB-468 tumor cells were co-cultured with untransduced T cells (Mock) or gD-CAR T cells for 24, 48, and 72 hours in the presence of indicated MOIs of T-VEC. As can be seen in FIG. 9, gD CAR T cells improved tumor cell killing.

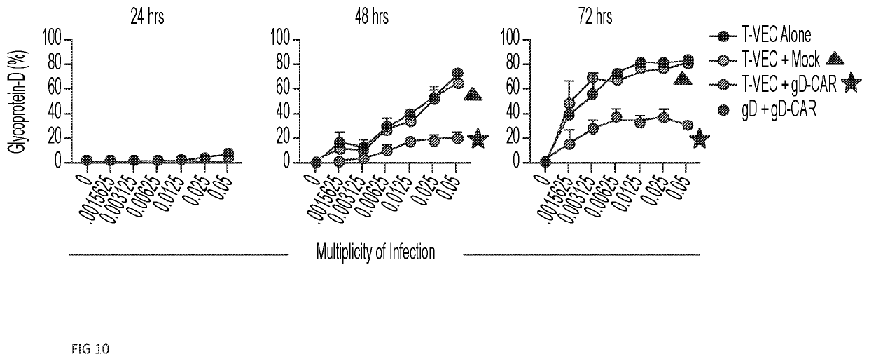

MDA-MB-468 tumor cells were co-cultured with untransduced T cells (Mock) or gD-CAR T cells for 24, 48, and 72 hours in the presence of indicated MOIs of T-VEC and gD expression was measured. As can be seen in FIG. 10, gD CAR T cells were specifically targeting T-VEC infected tumor cells expressing gD.

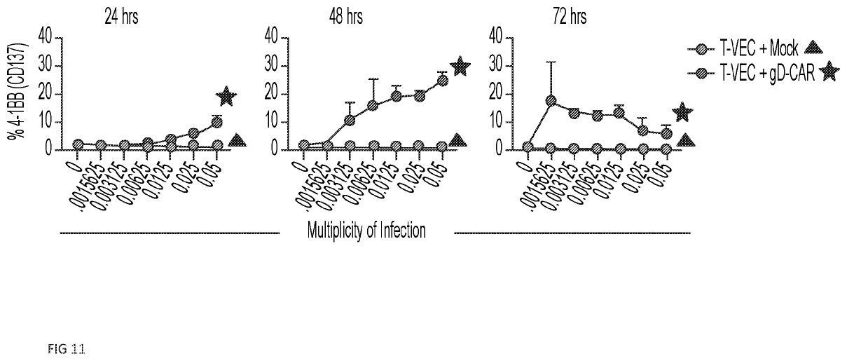

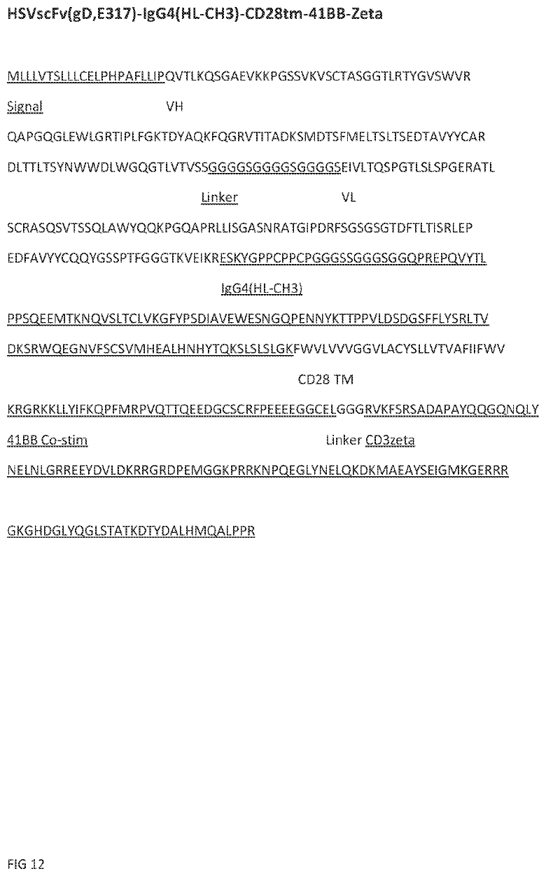

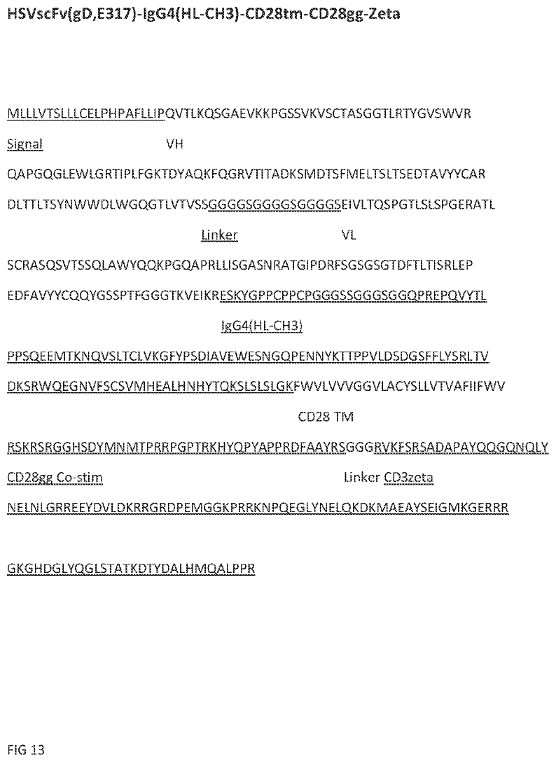

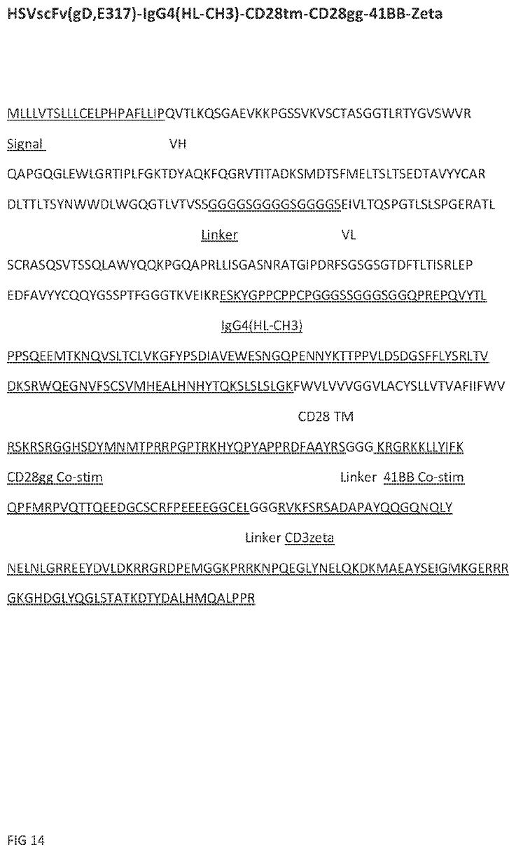

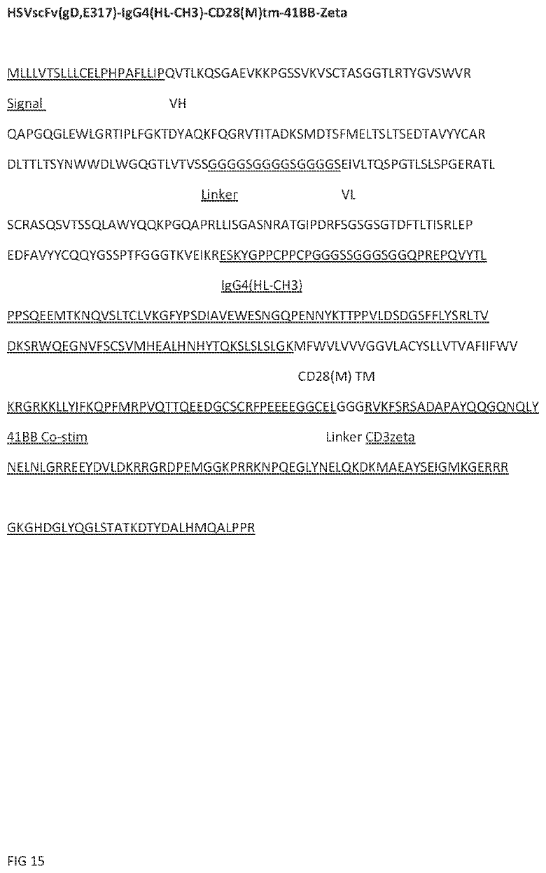

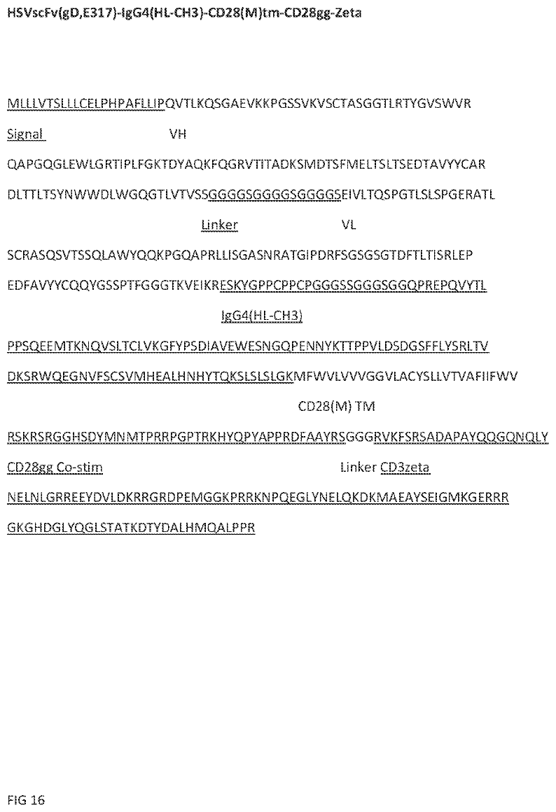

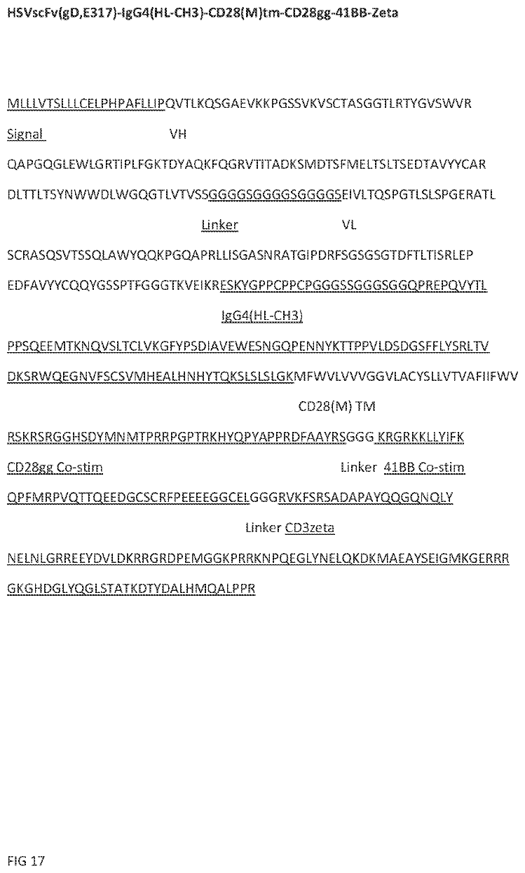

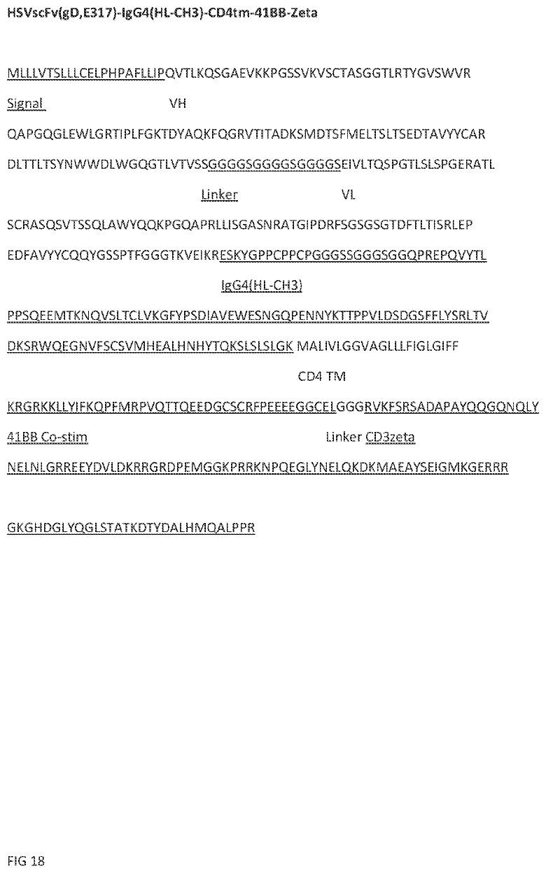

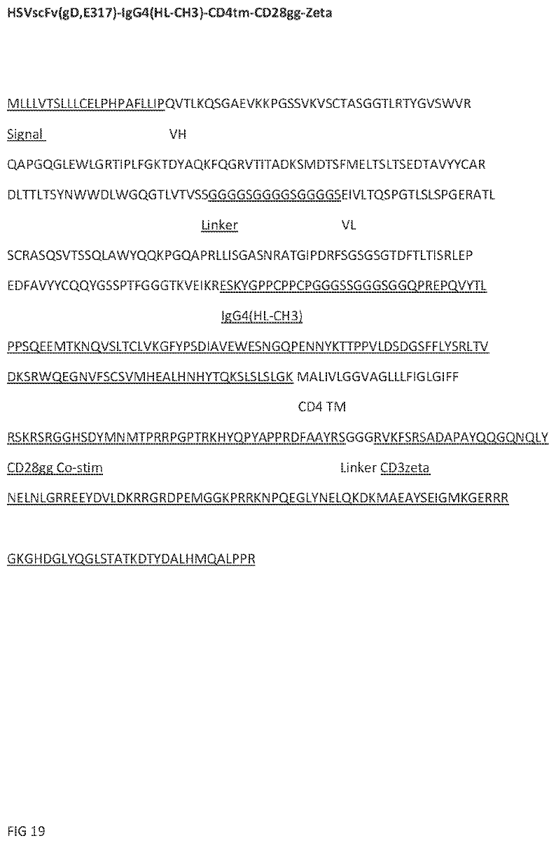

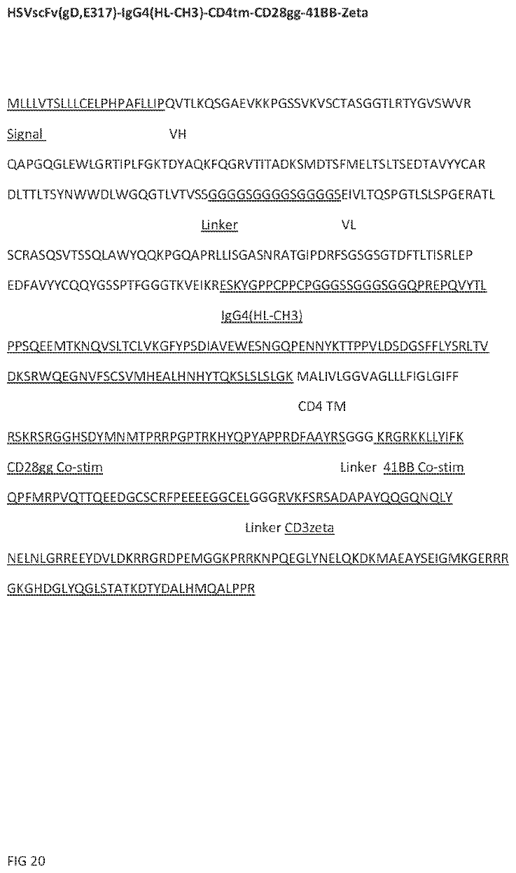

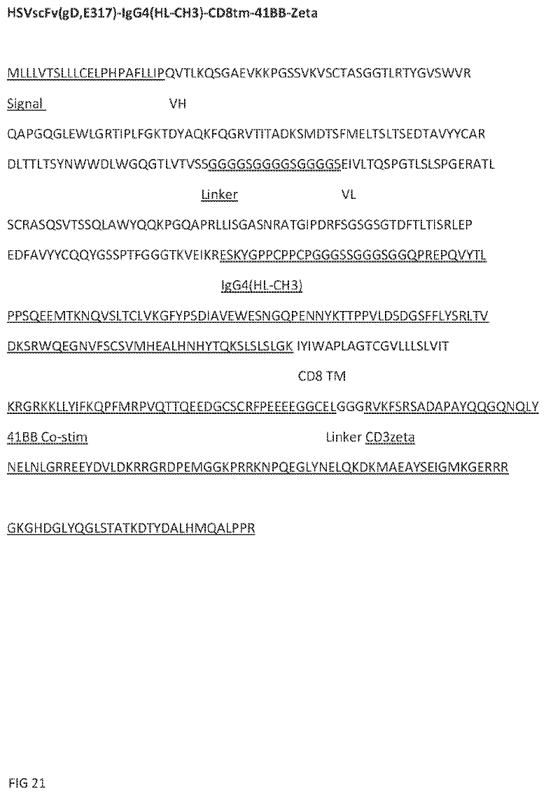

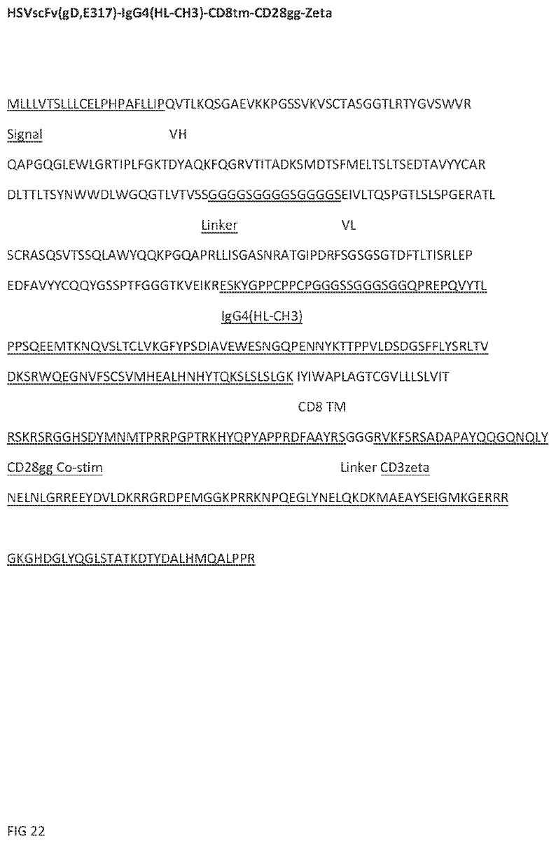

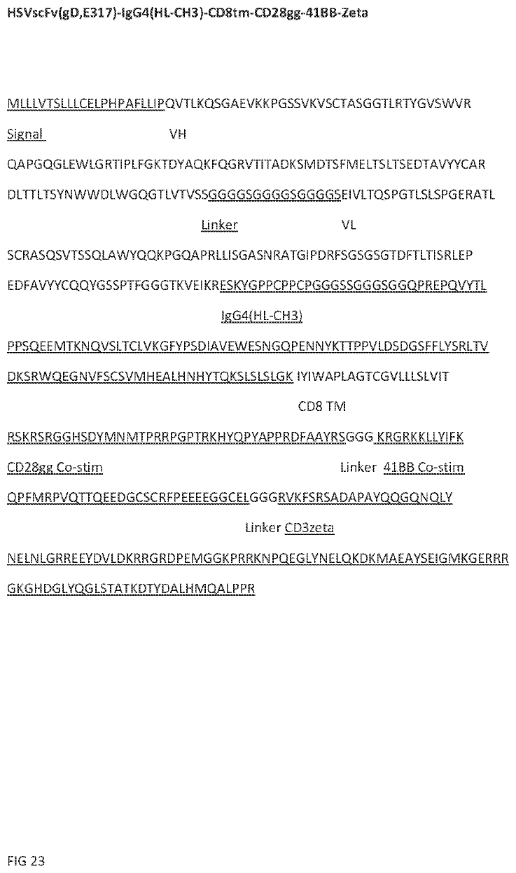

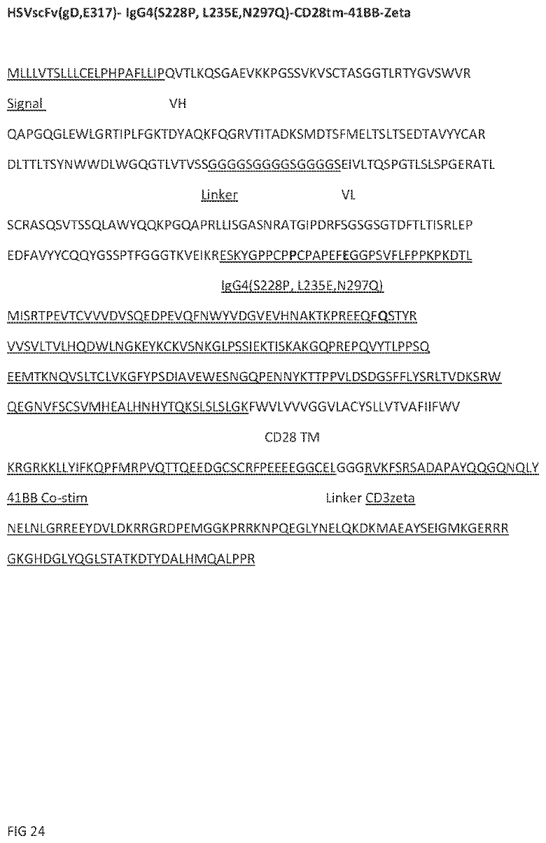

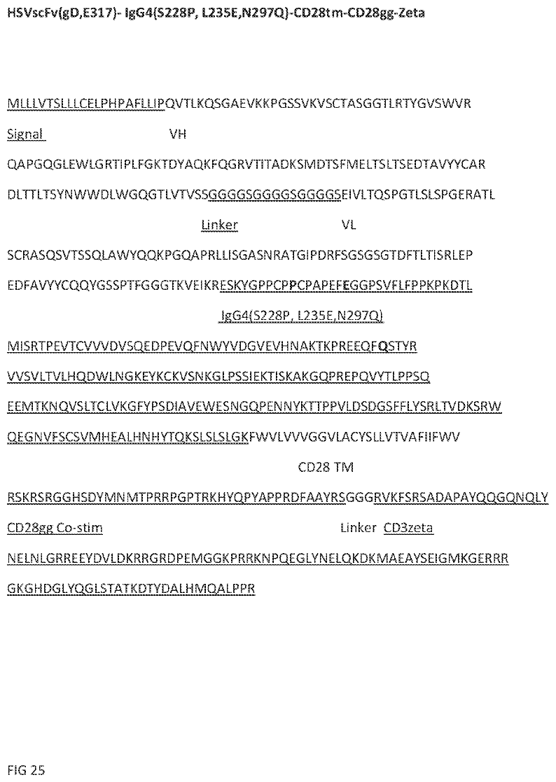

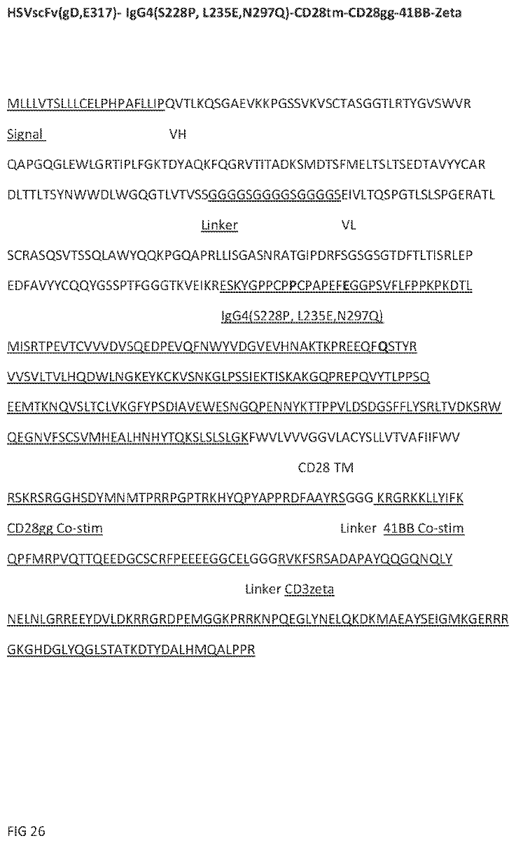

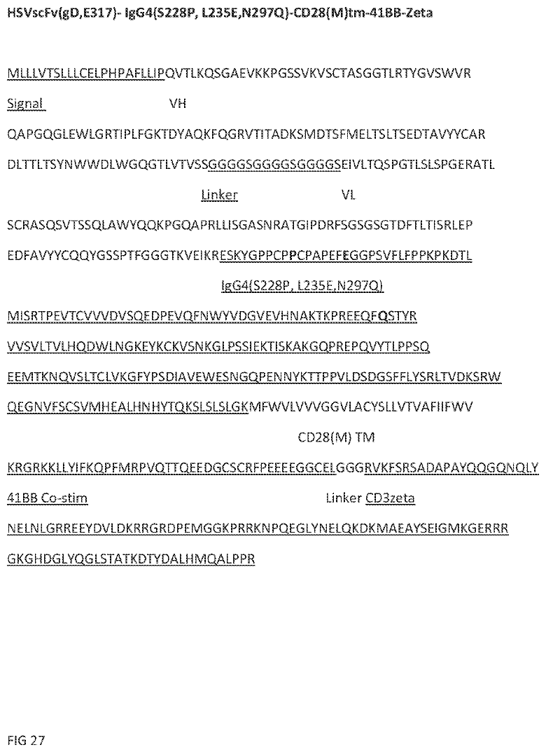

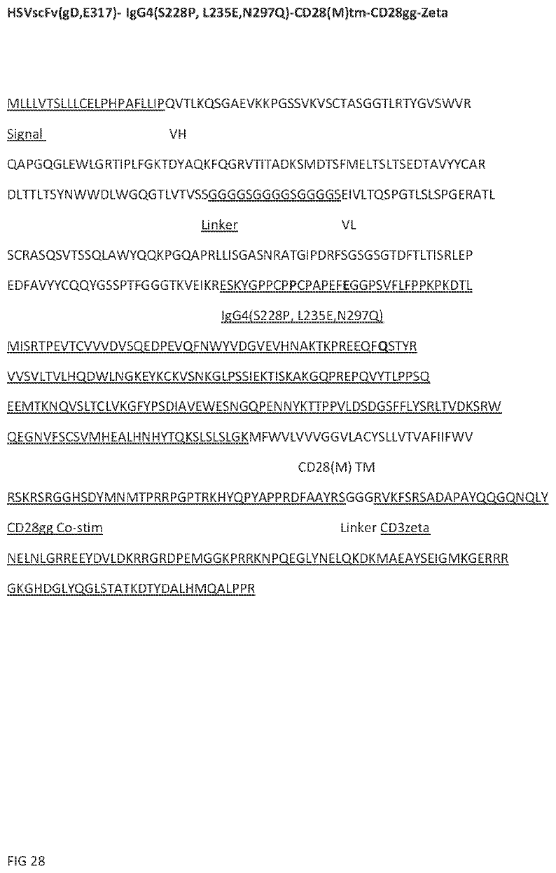

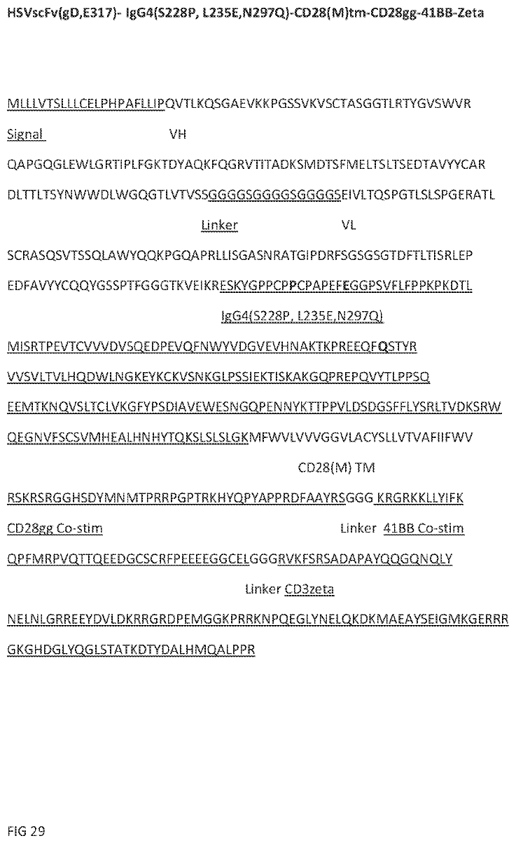

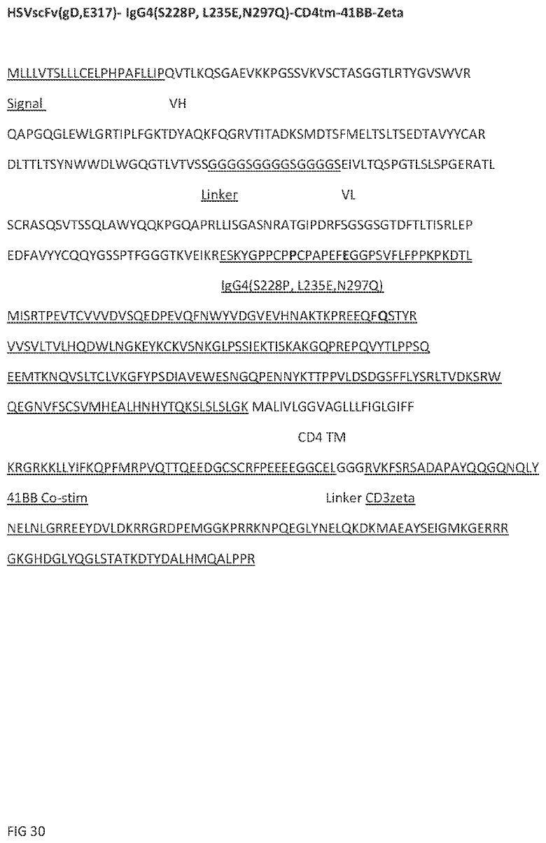

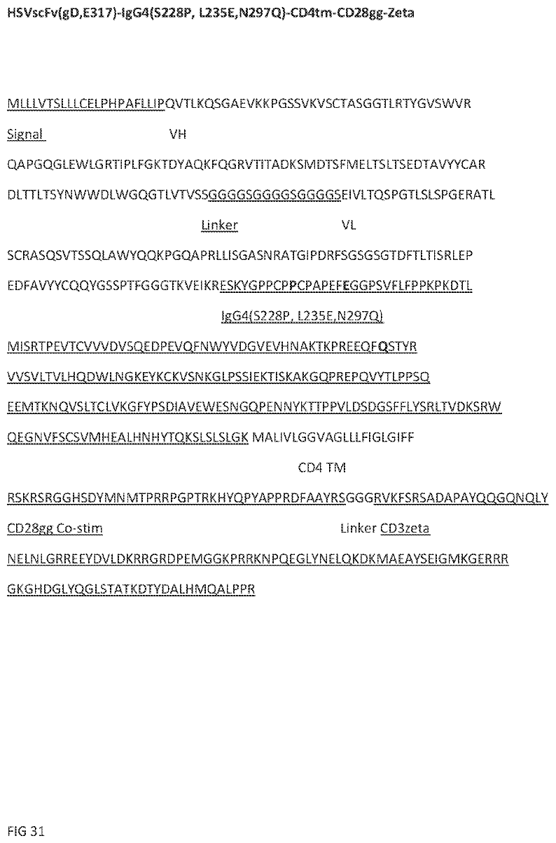

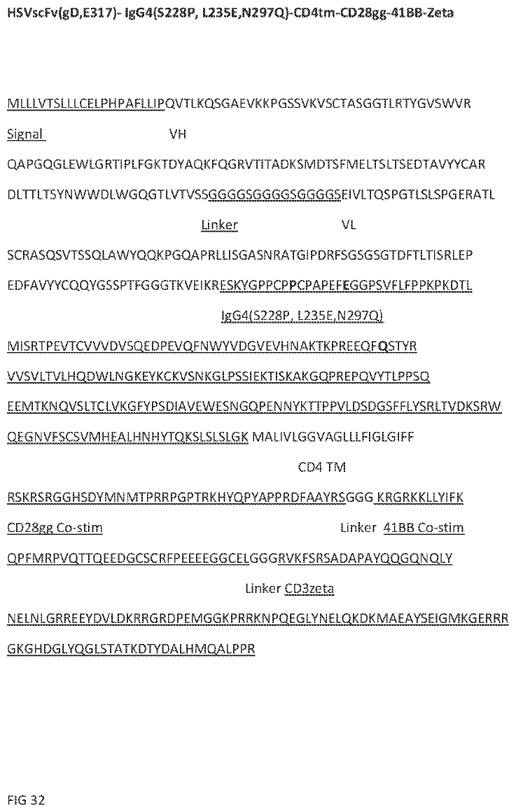

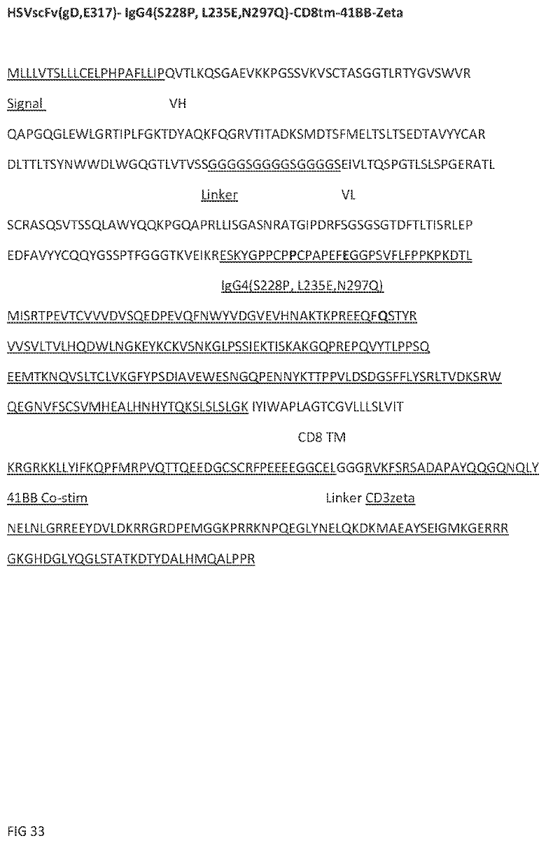

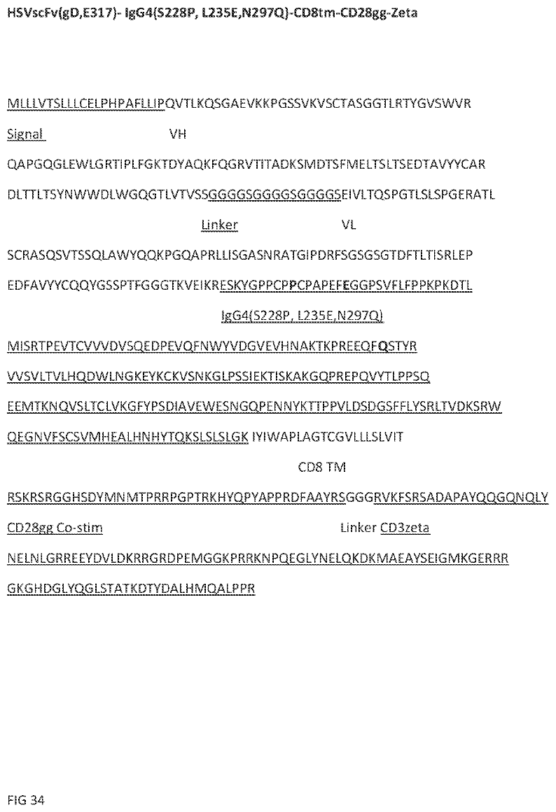

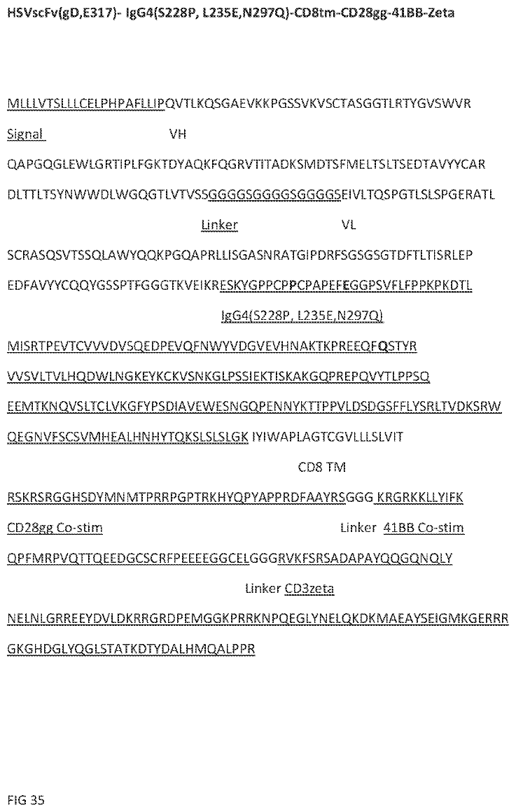

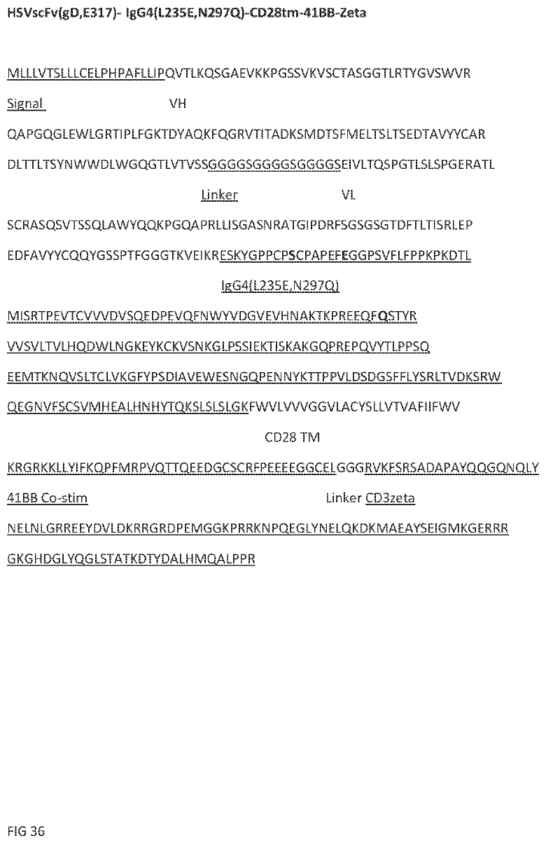

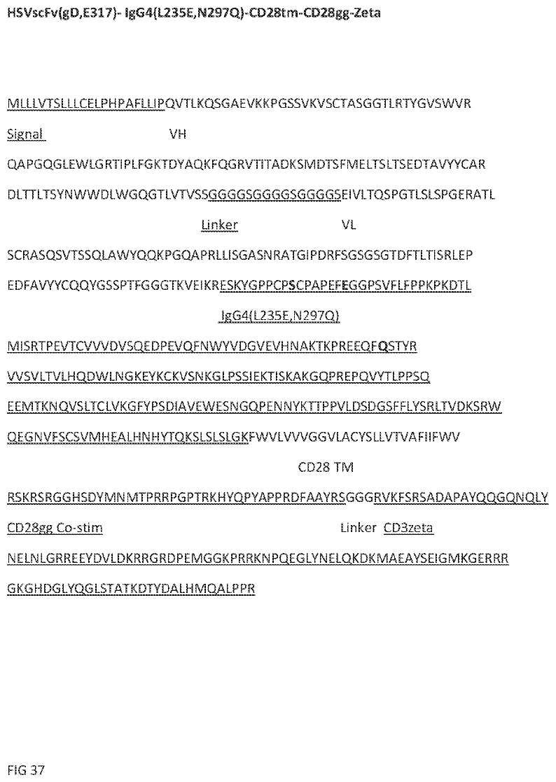

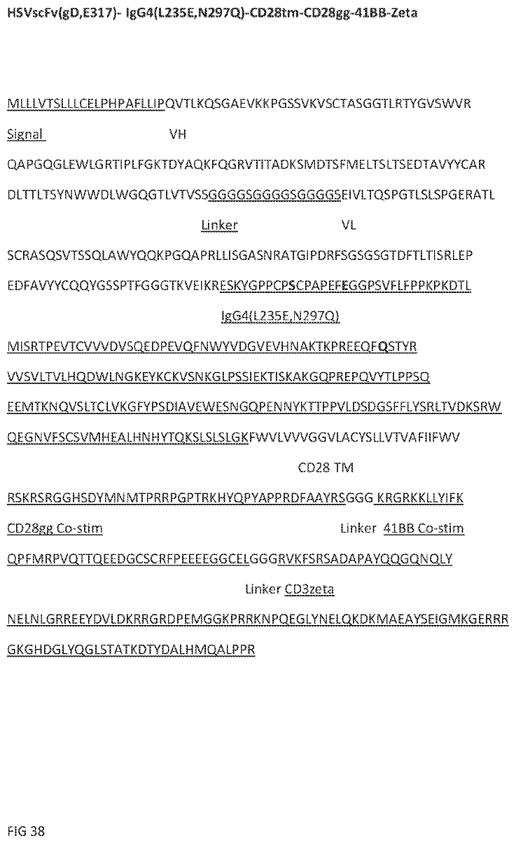

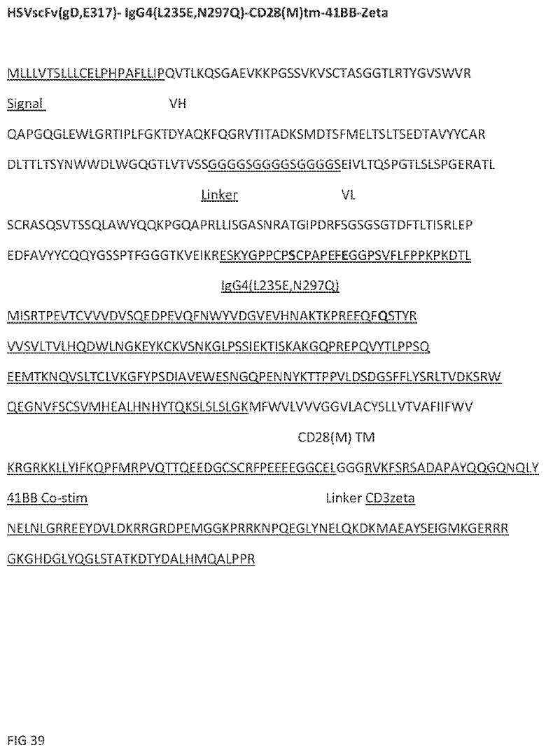

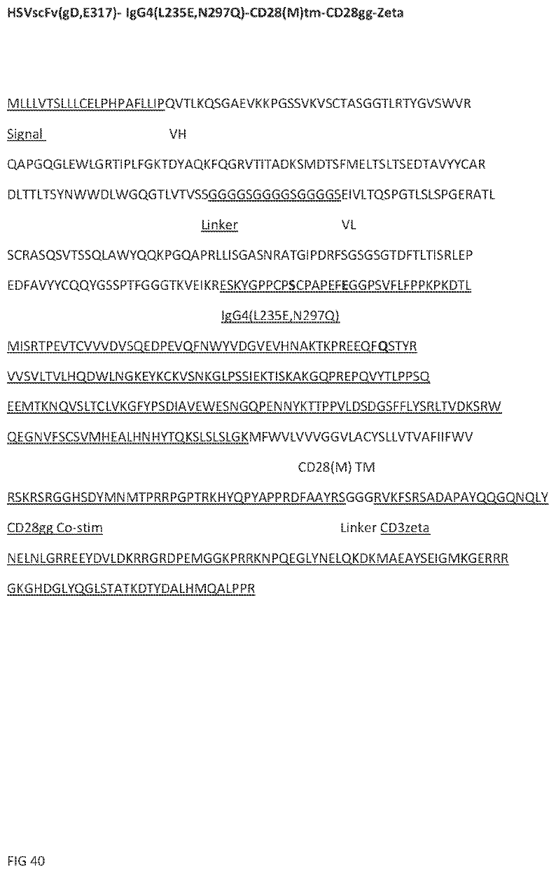

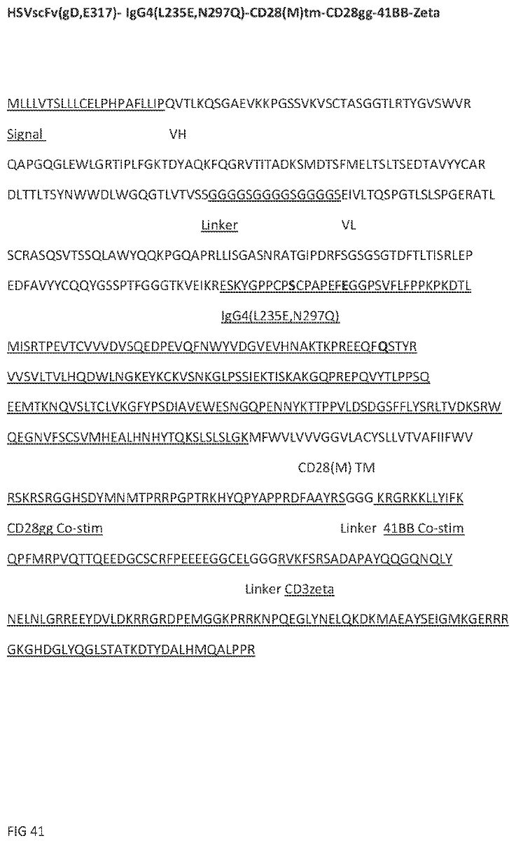

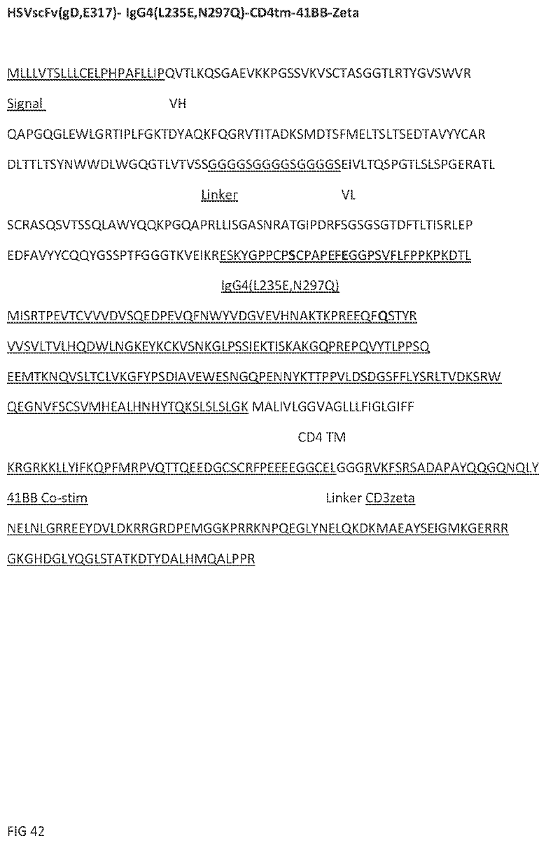

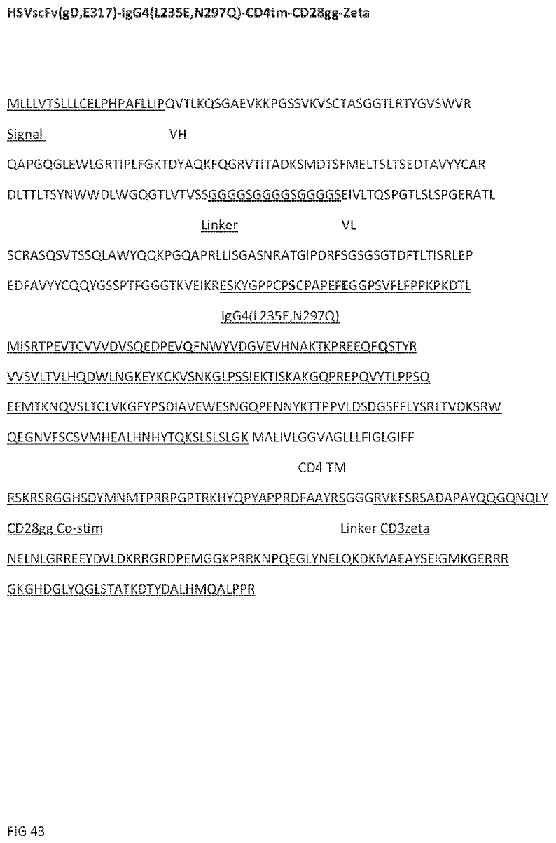

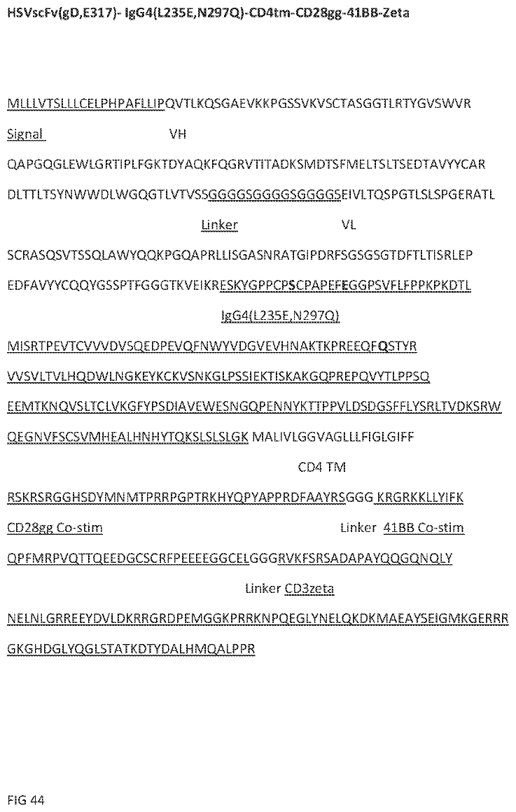

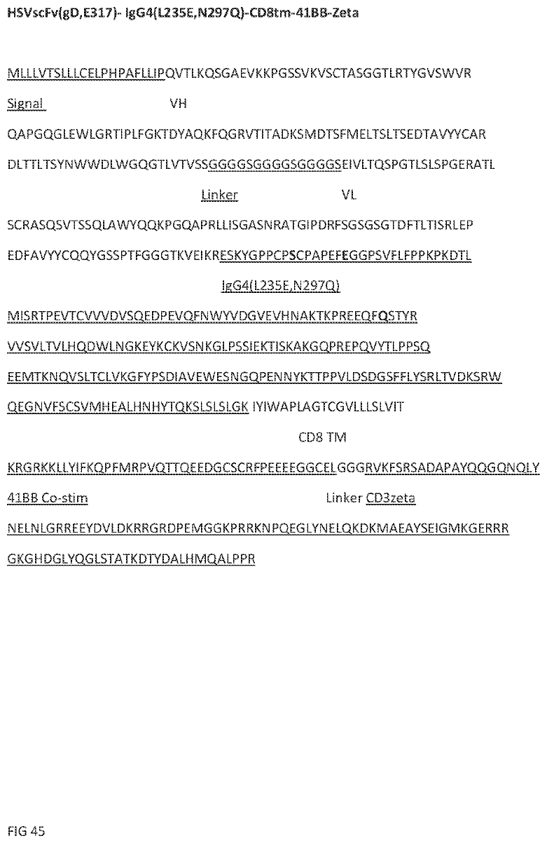

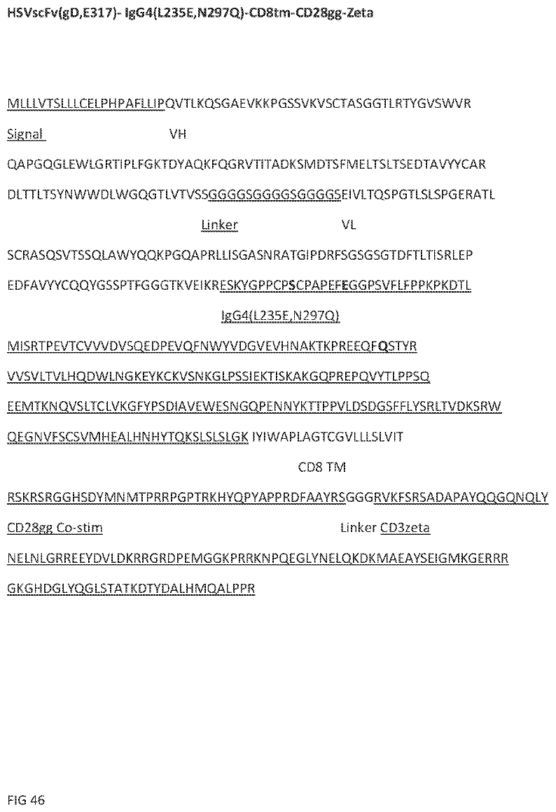

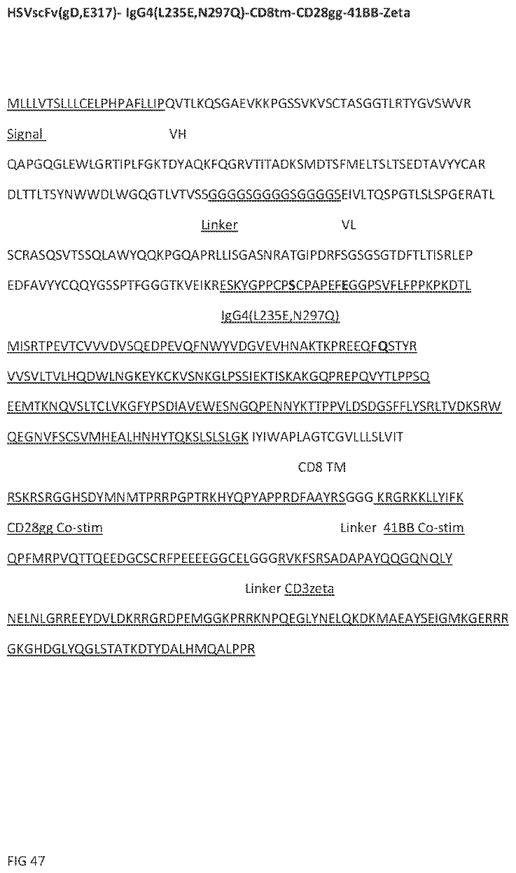

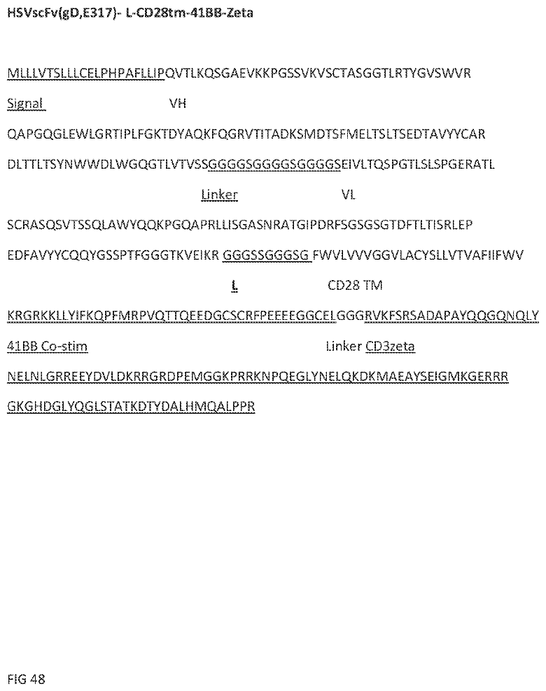

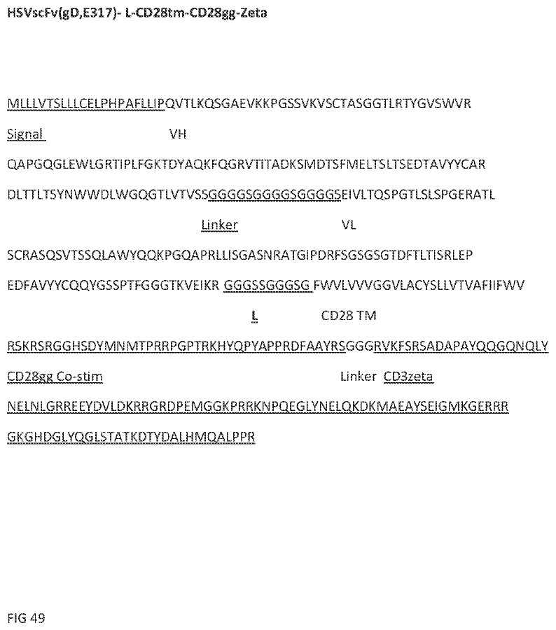

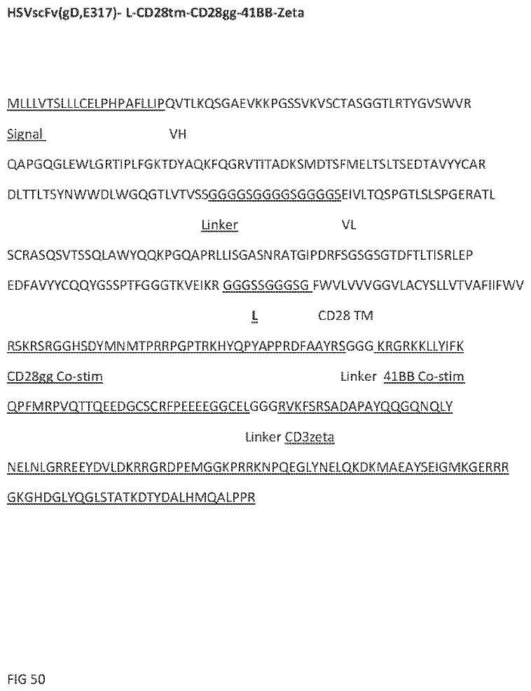

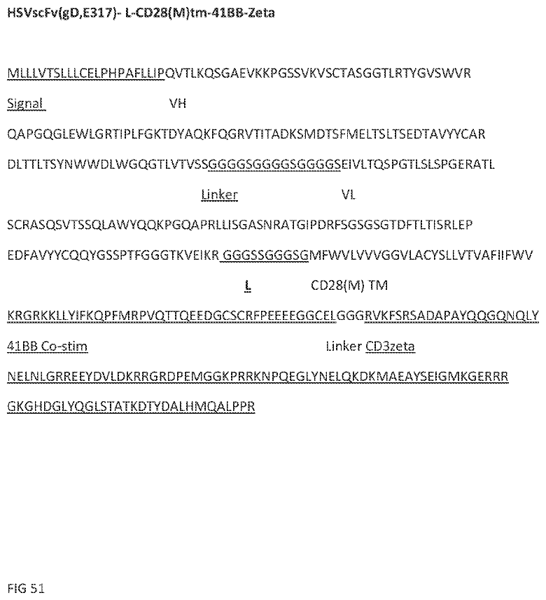

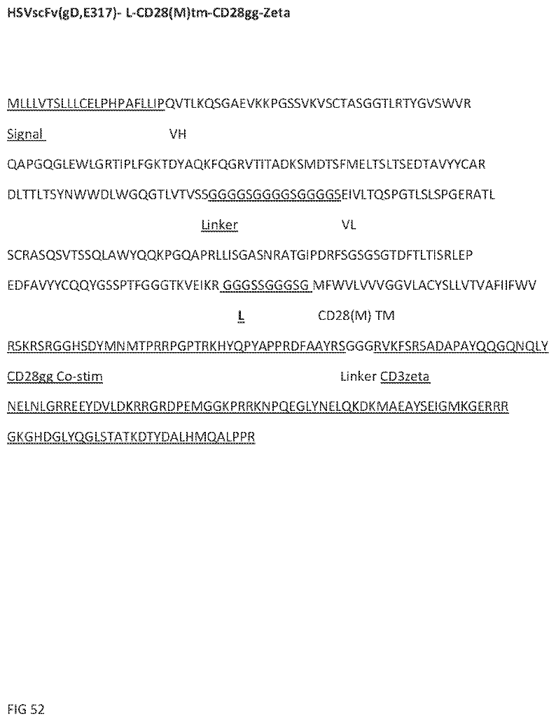

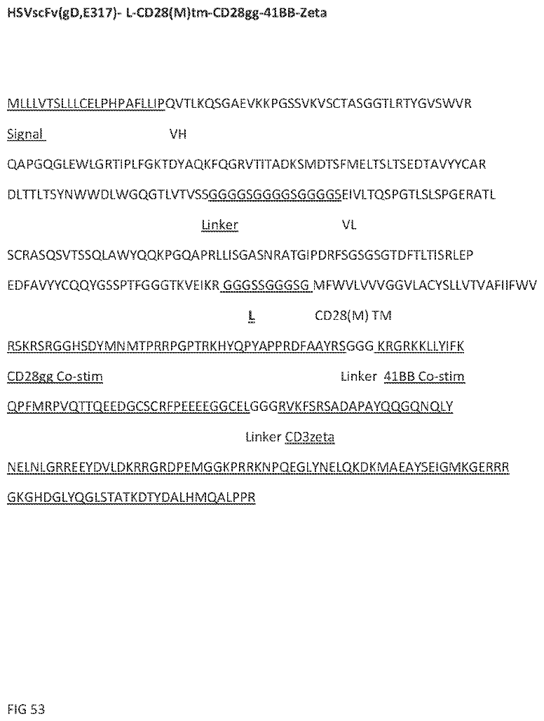

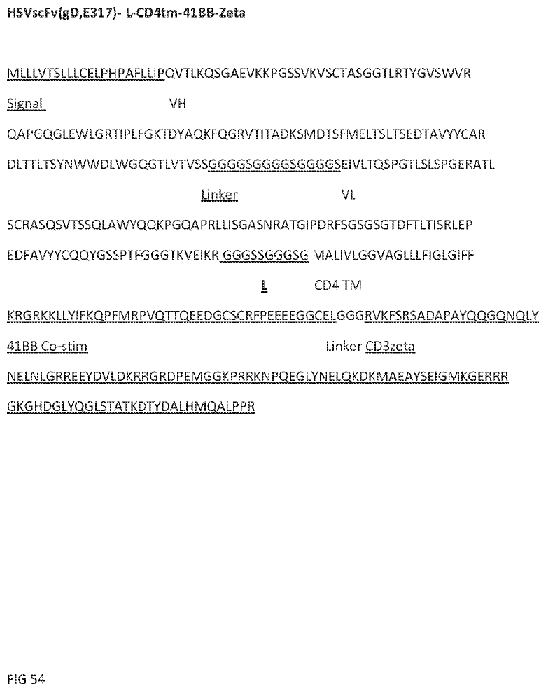

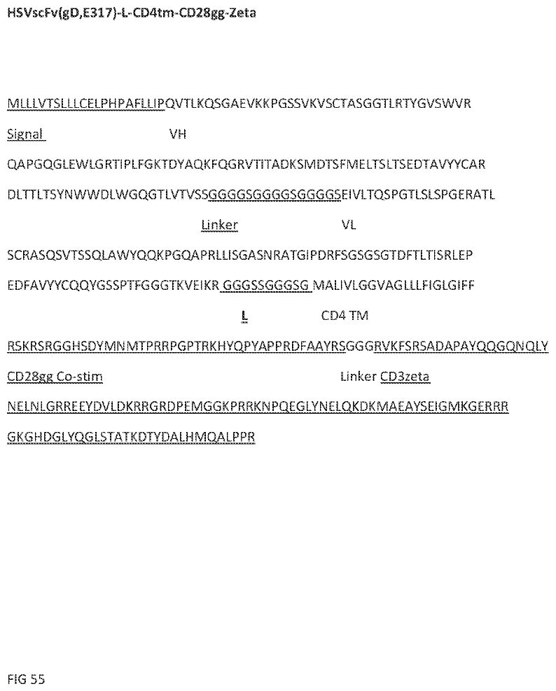

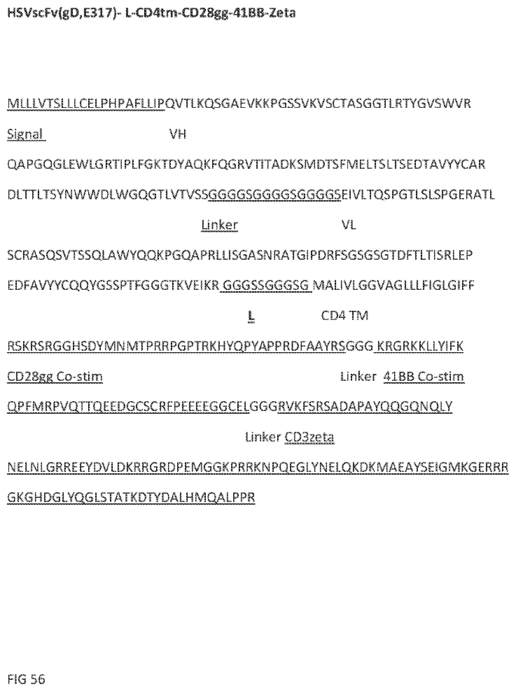

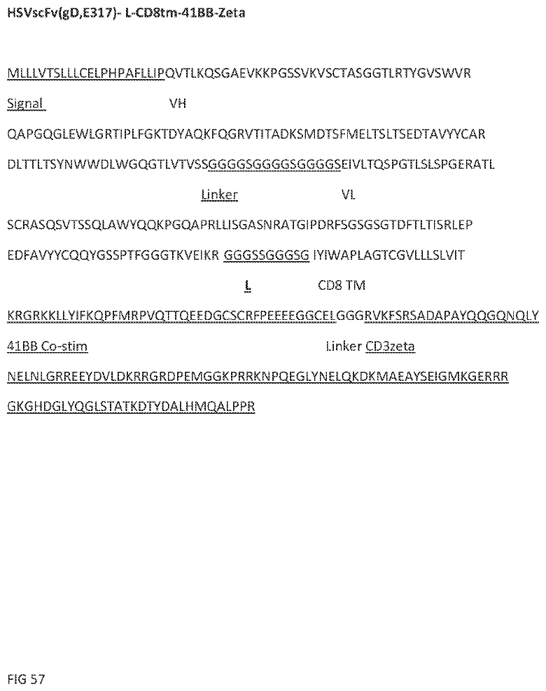

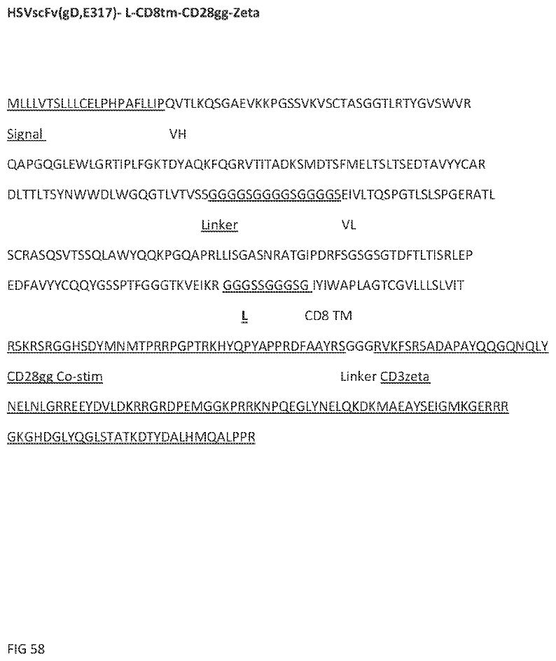

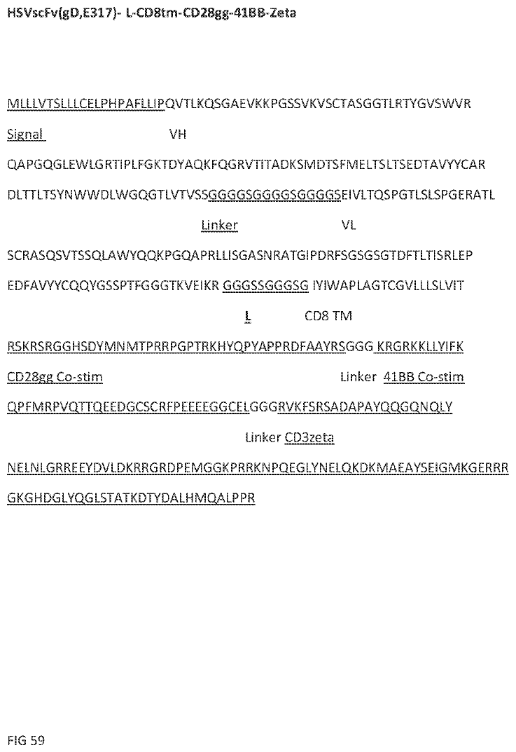

CD137 expression by mock transfected T cells and gD CAR T cells was measured following 24, 48 or 72 hour co-culture with MDA-MB-468 tumor cells or MDA-MB-468 tumor cells with T-VEC at various MOI. As can be seen in FIG. 11, gD CAR T cells were being activated against T-VEC infected tumor cells expressing gD.

### Example 6: TVEC Infected and Induced Surface Expression of Glycoprotein D onto Solid Mouse Tumors In Vitro

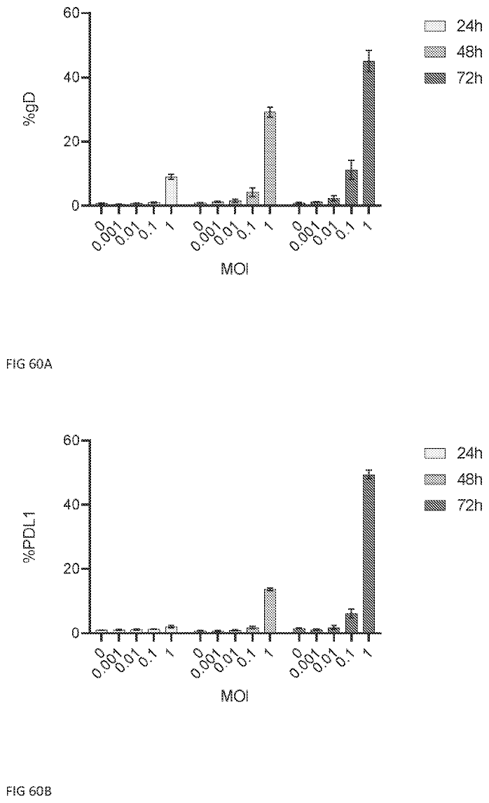

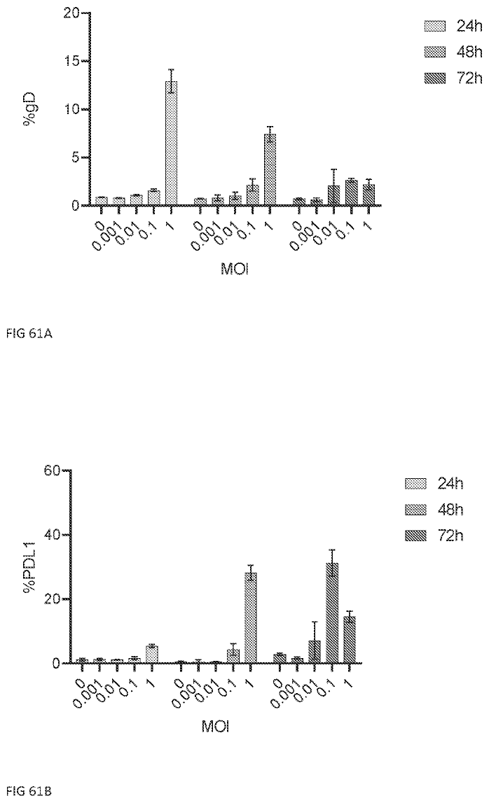

Flow cytometric analysis using a 96 well plate and 25,000 tumor cells/well showed MOI dependent increase in percent gD (FIG. 60A) and PDL1 expression (FIG. 60B) on MC38 tumor cells following 24-, 48-, and 72-hour co-culture with TVEC. The assay also showed MOI dependent increase in percent gD (FIG. 61A) and PDL1 expression (FIG. 61B) on EMT6 tumor cells following 24-, 48-, and 72-hour co-culture with TVEC.

### Example 7: Expression of the gD-CAR and Expansion of gD-mCAR T cells

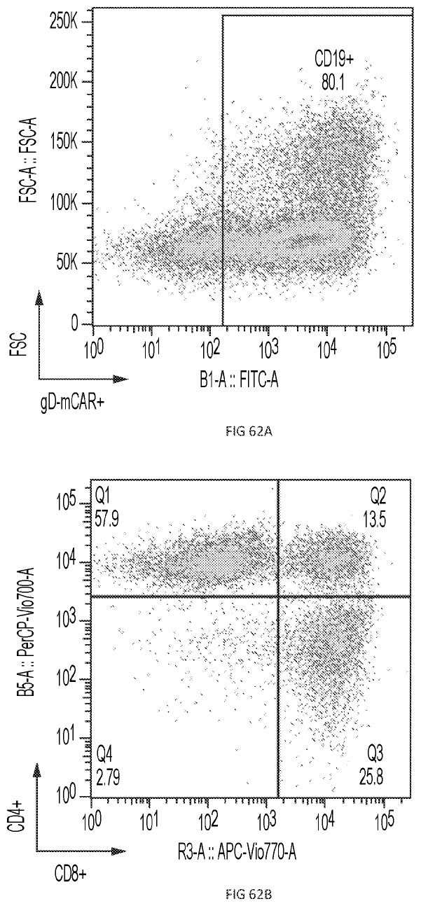

FACS plots of murine D-CAR T cells showed at least 80% of the T cells were successfully transduced and expressed the CAR (FIG. 62A). A FACS plot of gD-CAR (detected via mCD 19 positivity) on the surface of ex vivo engineered enriched murine T cells infected with retrovirus carrying the gD-CAR construct. A FACS plot of CD4+ and CD8+ population distributions of gD-mCAR expressing T cells (FIG. 62B).

### Example 8: TVEC Introduces gD on Murine Tumor Cells, which Directs Cytotoxicity and Activation of Murine gD-CAR T Cells In Vitro

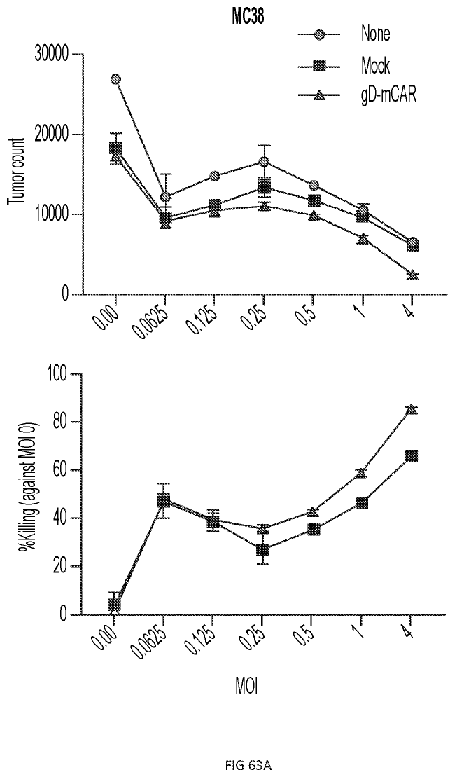

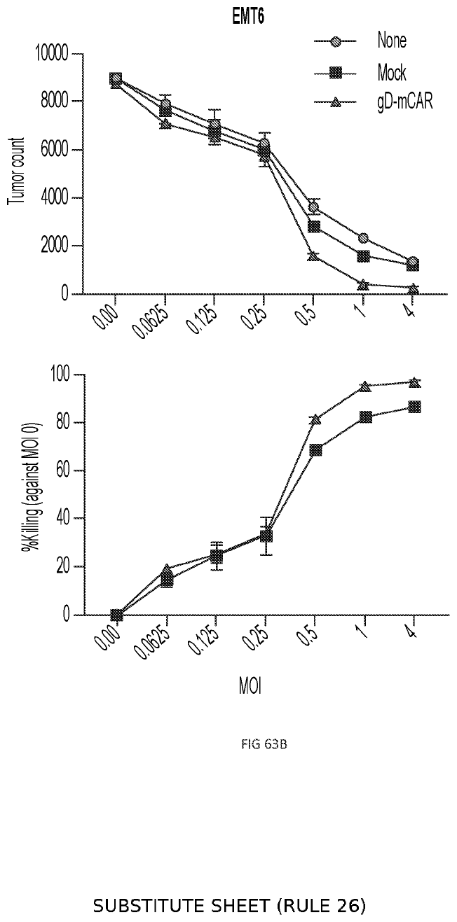

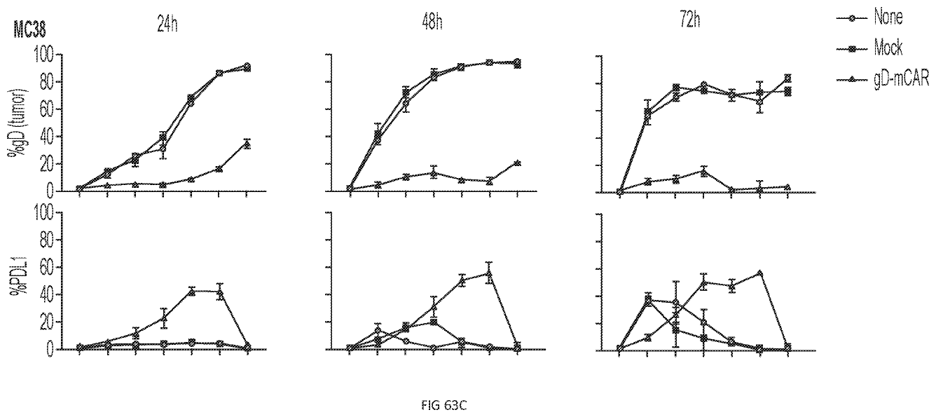

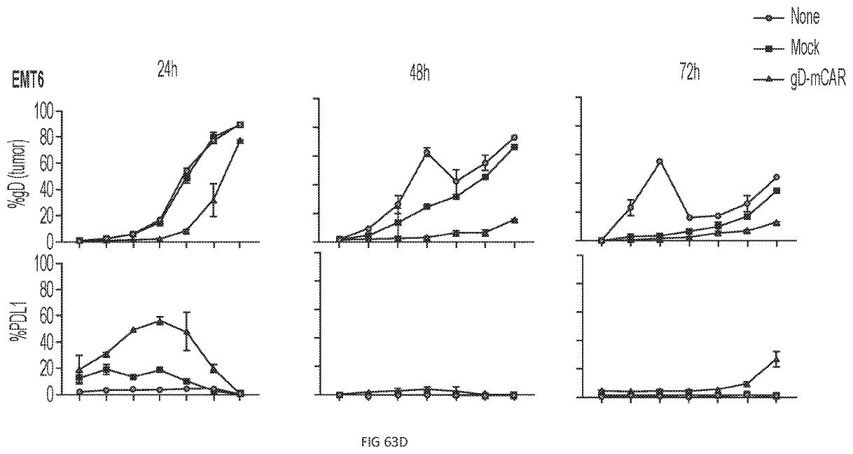

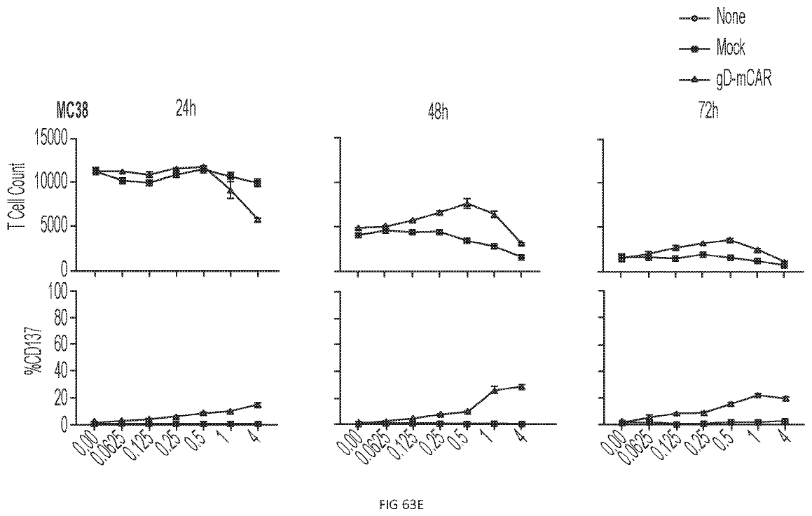

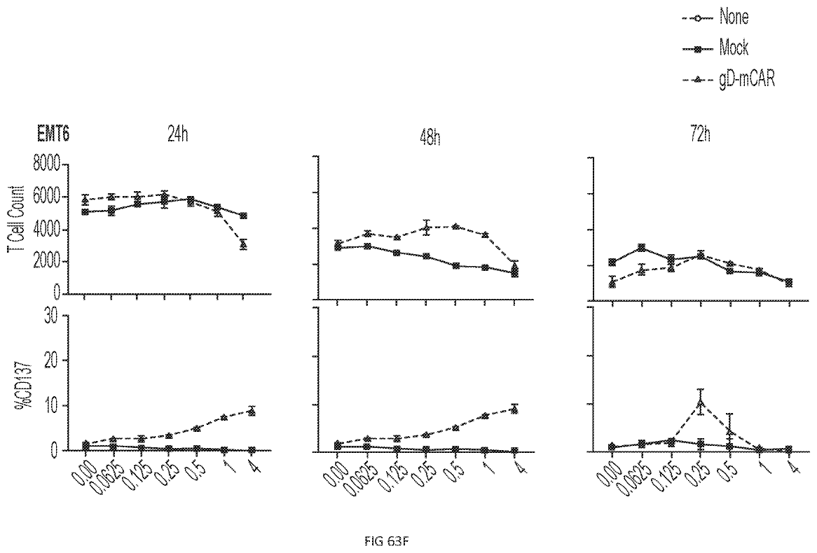

The mouse tumor cell killing ability of treatment with TVEC and a gD-mCAR was assessed by flow cytometry. MC38 cells (20,000 cells/well; FIG. 63A) and EMT6 cells (10,000 cells/well; FIG. 63B) were cocultured with TVEC at the indicated MOIs with untransduced T cells (mock) or gD-mCAR T cells at a 1:1 E:T ratio. The combination of TVEC treatment and gD-mCAR T cells treatment led to decreased tumor cell count and increased killing of tumor cells.

Percent of MC38 cells (FIG. 63C) and EMT6 cells (FIG. 63D) positive for gD in killing assay also show the increased tumor killing due to combination of TVEC treatment and gD-mCAR T cells treatment. Percent of MC38 cells (FIG. 63C) and EMT6 cells (FIG. 63D) positive PDL1 in killing were also assessed by flow cytometry at the indicated time points.

The activation of gD-mCAR T cells against TVEC-infected tumor cells (MC38 cells (FIG. 63E) and EMT6 cells (FIG. 63F)) expressing gD was also assessed. Quantification of T cell count and percent CD137 expression on untransduced T cells (mock) or gD-mCAR T cells in the killing assay was assessed by flow cytometry at the indicated time points. The combination of TVEC+gD-mCAR T-cells had better expansion and activation than that of the TVEC+Mock T cells.

The above data are from duplicate wells from one experiment and shown as means+SEM.

### Example 9: TVEC and gD-CAR T cell therapy showed potent antitumor efficacy

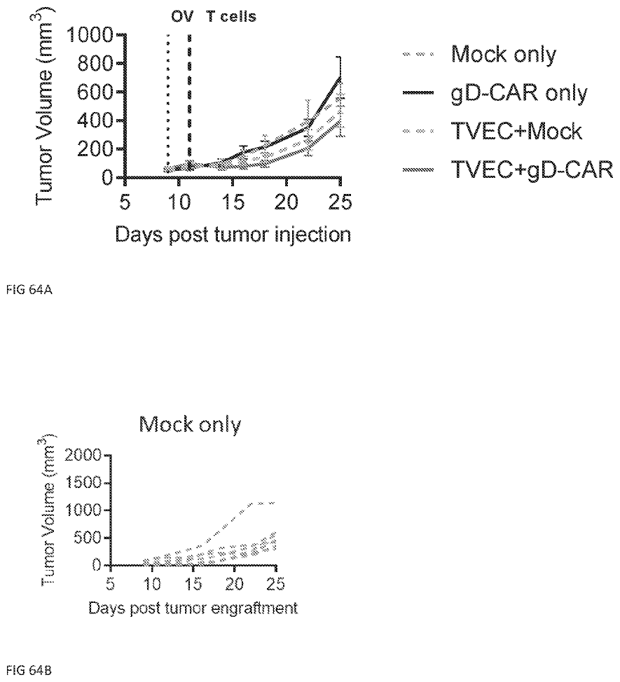

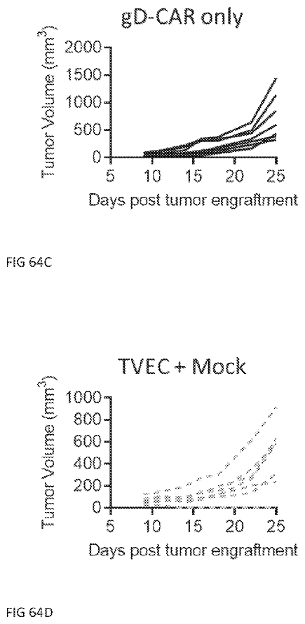

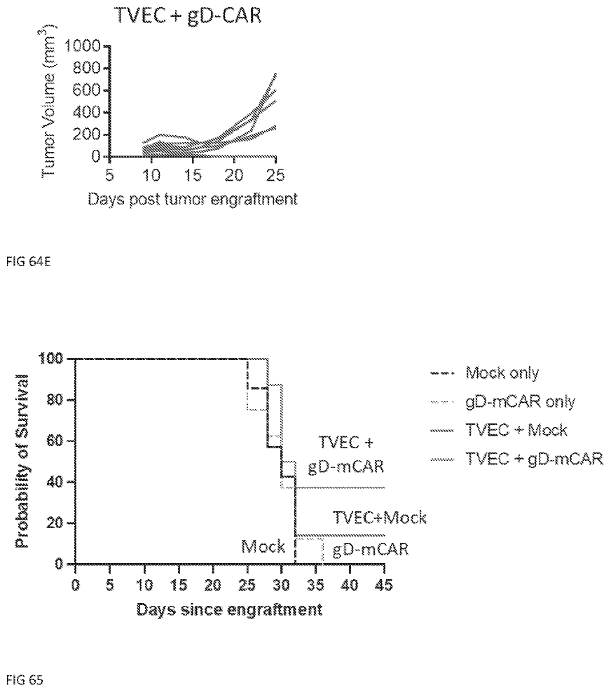

C57BL/Bj mice were engrafted with subcutaneous (s.c.) MC38 tumors (5×10⁵ cells) and on day 8 were treated with intraperitoneal cyclophosphamide, and subsequently treated intratumorally (i.t.) with 5×107 plaque forming units (pfu) per mice per day on days 9 and 10. On day 11, mice were treated with murine gD-CAR T cells intratumorally. Tumor volumes were measured with calipers. The data showed the antitumor efficacy of combination therapy of TVEC and murine gD-CAR T cells in an immunocompetent murine syngeneic tumor model (FIG. 64A). Table 4 below shows the treatment particulars.

TABLE 4

| | | | Tc | CPA | | Tumor |
|---|---|---|---|---|---|---|
| Group | Tumor | n | Treatment | treatment | oHSV | Harvest |
| 1 | MC38 | 8 | Mock | 100 mg/kg | None | 1 day post |
| | | | | | | TVEC |
| | | | | | | (n = 3) |
| 2 | MC38 | 8 | mgD-CAR | 100 mg/kg | None | 1 day post |
| | | | | | | TVEC |
| | | | | | | (n = 3) |
| 3 | MC38 | 8 | Mock | 100 mg/kg | TVEC | 1 day post |
| | | | | | (5 × 10⁷) × | TVEC |
| | | | | | 2 doses | (n = 3) |
| 4 | MC38 | 8 | mgD-CAR | 100 mg/kg | TVEC | 1 day post |
| | | | | | (5 × 10⁷) × | TVEC |
| | | | | | 2 doses | (n = 3) |

Data for each mouse in each group are also shown (mock only, FIG. 64B; gD-CAR only, FIG. 64C; TVEC+mock, FIG. 64D; and TVEC+gD CAR treatment, FIG. 64E).

The Kaplan-Meier survival curves confirm that the group receiving the TVEC and gD-mCAR T cells treatment had superior survival over the other groups (FIG. 65).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid molecule encoding a chimeric antigen receptor, wherein the chimeric antigen receptor comprises:
(i) an scFv that binds HSV envelope glycoprotein D, wherein the scFv comprises: a light chain CDR1 comprising RASQSVTSSQLA, a light chain CDR2 comprising GASNRAT, a light chain CDR3 comprising QQYGSSPT, a heavy chain CDR1 comprising TYGVS or GGTLRTYGVS, a heavy chain CDR2 comprising RTIPLFGKTDYAOKFOG, and a heavy chain CDR3 comprising DLTTLTSYNWWDL;
(ii) a spacer domain comprising a sequence selected from the group consisting of: SEQ ID NOs: 24-34;
(iii) a transmembrane domain comprising a sequence selected from the group consisting of SEQ ID NOs: 15-23;
(iv) a costimulatory domain comprising a sequence selected from the group consisting of SEQ ID NOs: 36-40; and
(v) a CD3ζ signaling domain.

2.-4. (canceled)

5. The nucleic acid molecule of claim 1, wherein the scFV comprises:
(a) a light chain variable domain that is at least 90%, 95%, 98%, or 100% identical to:
EIVLTQSPGTLSLSPGERATLSCRASQSVTSSQLAWYQQKPGQAPRLLIS
GASNRATGIPDRFSGSGSGTDFTLTISRLEPEDFAVYYCQQYGSSPTFGG
GTKVEIKR;
(b) a heavy chain variable domain that is at least 90%, 95%, 98%, or 100% identical to:
QVTLKQSGAEVKKPGSSVKVSCTASGGTLRTYGVSWVRQAPGQGLEWLGRTIPLFGKT DYAQKFQGRVTITADKSMDTSFMELTSLTSEDTAVYYCARDLTTLTSYNWWDLWGQG TLVTVSS; or
(c) a light chain variable domain that is at least 90%, 95%, 98%, or 100% identical to:
EIVLTQSPGTLSLSPGERATLSCRASQSVTSSQLAWYQQKPGQAPRLLISGASNRATGIPD RFSGSGSGTDFTLTISRLEPEDFAVYYCQQYGSSPTFGGGTKVEIKR; and
a heavy chain variable domain that is at least 90%, 95%, 98%, or 100% identical to:
QVTLKQSGAEVKKPGSSVKVSCTASGGTLRTYGVSWVRQAPGQGLEWLGR
TIPLFGKTDYAQKFQGRVTITADKSMDTSFMELTSLTSEDTAVYYCARDL
TTLTSYNWWDLWGQGTLVTVSS.

6.-7. (canceled)

8. The nucleic acid molecule of claim 1, wherein the scFv comprises SEQ ID NO: 2.

9.-10. (canceled)

11. The nucleic acid molecule of claim 1, wherein the chimeric antigen receptor comprises the amino acid sequence of SEQ ID NO: 48-49 or 57-126.

12. An immune cell harboring the nucleic acid molecule of claim 1.

13. A method of treating a patient infected with HSV, the method comprising administering a therapeutically effective amount of cells of claim 12.

14. A method of treating cancer, comprising administering an oncolytic HSV (oHSV) and a therapeutically effective amount of the cells of claim 12.

15. The method of claim 14, wherein the oHSV: lacks a functional ICP34.5 encoding gene; lacks a functional ICP47 encoding gene; and comprises a gene encoding human GM-CSF.

16. The method of claim 14, wherein the oHSV is talimogene laherparepvec.

17. The method of claim 14, wherein the oHSV is selected from the group consisting of: HF-10 (Takara Bio, Inc.; lacks UL43, UL49.5, UL55, UL56, and LAT), HSV-1716 (Virttu Biologics; lacks ICP34.5), G207 (Medigene; lacks ICP34.5 and ICP6 (substituted with LacZ), M032 (Acttis, Inc), and G47A (Daiichi Sankyo Company; lacks ICP34.5, ICP6 and ICP47).

18. The method of claim 14, further comprising an effective amount of an anti-PD-1 antibody or anti-CTLA-4 antibody.

19. The method of claim 18, wherein the anti-PD-1 antibody is selected from the group consisting of nivolumab, lambrolizumab, CT-011, and AMP-224.

20. The method of claim 18, wherein the anti-CTLA-4 antibody is ipilimumab.

21. A chimeric antigen receptor comprising:
(i) an scFv that binds HSV envelope glycoprotein D, wherein the scFv comprises: a light chain CDR1 comprising RASQSVTSSQLA, a light chain CDR2 comprising GASNRAT, a light chain CDR3 comprising QQYGSSPT, a heavy chain CDR1 comprising TYGVS or GGTLRTYGVS, a heavy chain CDR2 comprising RTIPLFGKTDYAQKFQG, and a heavy chain CDR3 comprising DLTTLTSYNWWDL;
(ii) a spacer domain comprising a sequence selected from the group consisting of: SEQ ID NOs: 24-34;
(iii) a transmembrane domain comprising a sequence selected from the group consisting of SEQ ID NOs: 15-23;
(iv) a costimulatory domain comprising a sequence selected from the group consisting of SEQ ID NOs: 36-40; and
(v) a CD3ζ signaling domain.

22. The chimeric antigen receptor of claim 21, wherein the scFV comprises: a light chain variable domain comprising
EIVLTQSPGTLSLSPGERATLSCRASQSVTSSQLAWYQQKPGQAPRLLISGASNRATGIPD RFSGSGSGTDFTLTISRLEPEDFAVYYCQQYGSSPTFGGGTKVEIKR; and
a heavy chain variable domain comprising
QVTLKQSGAEVKKPGSSVKVSCTASGGTLRTYGVSWVRQAPGQGLEWLGRTIPLFGKT DYAQKFQGRVTITADKSMDTSFMELTSLTSEDTAVYYCARDLTTLTSYNWWDLWGQG TLVTVSS.

23.-25. (canceled)

26. The chimeric antigen receptor of claim 21, wherein the chimeric antigen receptor comprises the amino acid sequence of any one of SEQ ID NO: 48-49 or 57-126.

27. A chimeric antigen receptor comprising a scFv comprising SEQ ID NO: 2; a spacer comprising a sequence selected from the group consisting of: SEQ ID NOs: 24-34; a transmembrane domain comprising a sequence selected from the group consisting of SEQ ID NOs: 15-23; a costimulatory domain comprising a sequence selected from the group consisting of SEQ ID NOs: 36-40, and a CD3 signaling domain comprising SEQ ID NO: 35.

28. An immune cell expressing the chimeric antigen receptor of claim 21.

29. A method of treating a patient infected with HSV, the method comprising administering a therapeutically effective amount of cells of claim 28.

30. A method of treating cancer, comprising administering an oncolytic HSV (oHSV) and a therapeutically effective amount of the cells of claim 28.


SEQUENCE LISTING (151 sequences)

SEQ ID NO: 1 - protein, 394 aa, Human alphaherpesvirus 1
MGGAAARLGAVILFVVIVGLHGVRGKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 2 - protein, 245 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKR

SEQ ID NO: 3 - protein, 22 aa, synthetic construct
MLLLVTSLLLCELPHPAFLLIP

SEQ ID NO: 4 - protein, 108 aa, synthetic construct [sequence omitted]

SEQ ID NO: 5 - protein, 12 aa, synthetic construct
RASQSVTSSQLA

SEQ ID NO: 6 - protein, 7 aa, synthetic construct
GASNRAT

SEQ ID NO: 7 - protein, 8 aa, synthetic construct
QQYGSSPT

SEQ ID NO: 8 - protein, 120 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTV

SEQ ID NO: 9 - protein, 5 aa, synthetic construct
TYGVS

SEQ ID NO: 10 - protein, 17 aa, synthetic construct
RTIPLFGKTDYAQKFQG

SEQ ID NO: 11 - protein, 13 aa, synthetic construct
DLTTLTSYNWWDL

SEQ ID NO: 12 - protein, 17 aa, synthetic construct
SSGGGGSGGGGSGGGGS

SEQ ID NO: 13 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 14 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 15 - protein, 21 aa, Homo sapiens
LCYLLDGILFIYGVILTALFL

SEQ ID NO: 16 - protein, 27 aa, Homo sapiens
FWVLVVVGGVLACYSLLVTVAFIIFWV

SEQ ID NO: 17 - protein, 28 aa, Homo sapiens
MFWVLVVVGGVLACYSLLVTVAFIIFWV

SEQ ID NO: 18 - protein, 22 aa, Homo sapiens
MALIVLGGVAGLLLFIGLGIFF

SEQ ID NO: 19 - protein, 21 aa, Homo sapiens
IYIWAPLAGTCGVLLLSLVIT

SEQ ID NO: 20 - protein, 23 aa, Homo sapiens
IYIWAPLAGTCGVLLLSLVITLY

SEQ ID NO: 21 - protein, 24 aa, Homo sapiens
IYIWAPLAGTCGVLLLSLVITLYC

SEQ ID NO: 22 - protein, 27 aa, Homo sapiens
IISFFLALTSTALLFLLFFLTLRFSVV

SEQ ID NO: 23 - protein, 21 aa, Homo sapiens
PFFFCCFIAVAMGIRFIIMVA

SEQ ID NO: 24 - protein, 10 aa, synthetic construct
GGGSSGGGSG

SEQ ID NO: 25 - protein, 12 aa, synthetic construct
ESKYGPPCPPCP

SEQ ID NO: 26 - protein, 12 aa, synthetic construct
ESKYGPPCPSCP

SEQ ID NO: 27 - protein, 22 aa, synthetic construct
ESKYGPPCPPCPGGGSSGGGSG

SEQ ID NO: 28 - protein, 39 aa, synthetic construct
IEVMYPPPYLDNEKSNGTIIHVKGKHLCPSPLFPGPSKP

SEQ ID NO: 29 - protein, 48 aa, synthetic construct
AKPTTTPAPRPPTPAPTIASQPLSLRPEACRPAAGGAVHTRGLDFACD

SEQ ID NO: 30 - protein, 45 aa, synthetic construct
TTTPAPRPPTPAPTIASQPLSLRPEACRPAAGGAVHTRGLDFACD

SEQ ID NO: 31 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 229 aa, synthetic construct
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 41 aa, Homo sapiens
RSKRSRLLHSDYMNMTPRRPGPTRKHYQPYAPPRDFAAYRS

SEQ ID NO: 37 - protein, 41 aa, synthetic construct
RSKRSRGGHSDYMNMTPRRPGPTRKHYQPYAPPRDFAAYRS

SEQ ID NO: 38 - protein, 42 aa, Homo sapiens
KRGRKKLLYIFKQPFMRPVQTTQEEDGCSCRFPEEEEGGCEL

SEQ ID NO: 39 - protein, 42 aa, Homo sapiens
ALYLLRRDQRLPPDAHKPPGGGSFRTPIQEEQADAHSTLAKI

SEQ ID NO: 40 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 41 - protein, 10 aa, synthetic construct
GGTLRTYGVS

SEQ ID NO: 42-44 - skipped (3 entries)

SEQ ID NO: 45 - protein, 24 aa, synthetic construct
LEGGGEGRGSLLTCGDVEENPGPR

SEQ ID NO: 46 - protein, 354 aa, synthetic construct
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 47 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 48 - protein, 580 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 558 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 51 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTYDALHMQALPPR

SEQ ID NO: 52 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 53 - protein, 112 aa, synthetic construct
RVKFSRSADAPAYQQGQNQLYNELNLGRREEYDVLDKRRGRDPEMGGKPRRKNPQEGLYNELQKDKMAEAFSEIGMKGERRRGKGHDGLFQGLSTATKDTFDALHMQALPPR

SEQ ID NO: 54 - protein, 112 aa, synthetic construct
RVKFSRSADAPAYQQGQNQLYNELNLGRREEYDVLDKRRGRDPEMGGKPRRKNPQEGLFNELQKDKMAEAYSEIGMKGERRRGKGHDGLFQGLSTATKDTFDALHMQALPPR

SEQ ID NO: 55 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTFDALHMQALPPR

SEQ ID NO: 56 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTYDALHMQALPPR

SEQ ID NO: 57 - protein, 579 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 557 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 624 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 602 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 581 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 559 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 580 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 558 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 625 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 603 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 575 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 553 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 574 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 552 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 619 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 597 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 574 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 552 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 573 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 551 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 618 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 596 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 680 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 658 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 679 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 657 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 724 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 702 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 681 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 659 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 680 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 658 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 725 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 703 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 675 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 653 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 674 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 652 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 719 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 697 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 674 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 652 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 673 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 651 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 718 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 696 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 680 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 658 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 679 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 657 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 724 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 702 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 681 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 659 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 680 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 658 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 725 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 703 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 675 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 653 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 674 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 652 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 719 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 697 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 674 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 652 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 673 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 651 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 718 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 696 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 461 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 439 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 460 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 438 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 505 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 483 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 462 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 440 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 461 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 439 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 506 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 484 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 456 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 434 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 455 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMALIVLGGVAGLLLFIGLGIFFRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 433 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMALIVLGGVAGLLLFIGLGIFFRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 500 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMALIVLGGVAGLLLFIGLGIFFRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 478 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGMALIVLGGVAGLLLFIGLGIFFRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 455 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 433 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 454 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGIYIWAPLAGTCGVLLLSLVITRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 432 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGIYIWAPLAGTCGVLLLSLVITRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 499 aa, synthetic construct
MLLLVTSLLLCELPHPAFLLIPQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGIYIWAPLAGTCGVLLLSLVITRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 477 aa, synthetic construct
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSSGGGGSGGGGSGGGGSEIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRGGGSSGGGSGIYIWAPLAGTCGVLLLSLVITRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 122 aa, synthetic construct [sequence omitted]